(19) United States
(12) Patent Application Publication
(10) Pub. No.: US 2012/0220779 A1
(43) Pub. Date: Aug. 30, 2012

(54) Novel Processes

(76) Inventors: Claire Frances Crawford, Stevenage (GB); Sandrine Garcia, Stevenage (GB); Jonathan Paul Graham, Stevenage (GB); Sandra Jane Harling, Stevenage (GB); Nicholas Paul Henley, Stevenage (GB); Stephen Andrew Hermitage, Stevenage (GB); John Howard Hutchinson, San Giego, CA (US); Trevor Raymond Keel, Stevenage (GB); Andrew Kennedy, Stevenage (GB); Andrew McMurtrie Mason, Stevenage (GB); Mark Simon Scott, Stevenage (GB); Neil Michael Smith, Stevenage (GB); Nicholas Simon Stock, San Diego, CA (US); Yuichi Tateno, Stevenage (GB); Leontine Saskia Trouw, Stevenage (GB); Peter Graham Turner, Stevenage (GB); Christopher John Wallis, Stevenage (GB); Robert David Willacy, Stevenage (GB)

(21) Appl. No.: 13/503,867

(22) PCT Filed: Nov. 2, 2010
(86) PCT No.: PCT/EP2010/066577

§ 371 Date: Apr. 25, 2012

Related U.S. Application Data
(60) Provisional application No. 61/257,479, filed on Nov. 3, 2009.
(60) Provisional application No. 61/260,453, filed on Nov. 12, 2009.
(60) Provisional application No. 61/366,178, filed on Jul. 21, 2010.

(51) Int. Cl. C07D 401/14

(52) U.S. Cl. 546/256

(57) ABSTRACT

The present invention provides processes useful for preparing 5-lipoxygenase activating protein (FLAP) inhibitors and their intermediates. In particular, processes for preparing 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid, the anhydrous Form C polymorph of sodium 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionate, and intermediates useful in said processes are provided.

### FIELD OF THE INVENTION

Described herein are processes useful for preparing 5-lipoxygenase activating protein (FLAP) inhibitors and their intermediates. In particular, described herein are processes for preparing 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid, the anhydrous Form C polymorph of sodium 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionate, and intermediates useful in said processes.

### BACKGROUND OF THE INVENTION

Leukotrienes are biological compounds formed from arachidonic acid in the leukotriene synthesis pathway. Leukotrienes are synthesized primarily by eosinophils, neutrophils, mast cells, basophils, dendritic cells, macrophages and monocytes. Leukotrienes have been implicated in biological actions including, by way of example only, smooth muscle contraction, leukocyte activation, cytokine secretion, mucous secretion, and vascular function.

FLAP is a member of the MAPEG (membrane associated proteins involved in eicosanoid and glutathione metabolism) family of proteins. FLAP is responsible for binding arachidonic acid and transferring it to 5-lipoxygenase. 5-Lipoxygenase can then catalyze the two-step oxygenation and dehydration of arachidonic acid, converting it into the intermediate compound 5-HPETE (5-hydroperoxyeicosatetraenoic acid), and in the presence of FLAP convert the 5-HPETE to Leukotriene A₄ (LTA₄). LTA₄ is converted to LTB₄ by LTA₄ hydrolase or, alternatively, LTA₄ is acted on by LTC₄ synthase, which conjugates LTA₄ with reduced glutathione (GSH) to form the intracellular product leukotriene C₄ (LTC₄). LTC₄ is transformed to leukotriene D₄ (LTD₄) and leukotrine E₄ (LTD₄) by the action of gamma-glutamyl-transpeptidase and dipeptidases. LTC₄ synthase plays a pivotal role as the only committed enzyme in the formation of cysteinyl leukotrienes.

Processes for preparing FLAP Inhibitors, in particular 3-[3-(tert-butylsulfanyl)-1[4-(6-ethoxy-pyridin-3-yl)benzyl]-5-(5-methyl-pyridin-2-yl-methoxy)-1H-indol-2-yl]-2,2-dimethyl-propionic acid, and intermediates useful in the synthesis of FLAP inhibitors, have been described in International patent application WO 2007/056021.

International patent application WO 2007/056021 describes a linear process for the preparation of FLAP inhibitors. In particular, WO 2007/056021 describes a process for the preparation of 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid via the following Scheme A:

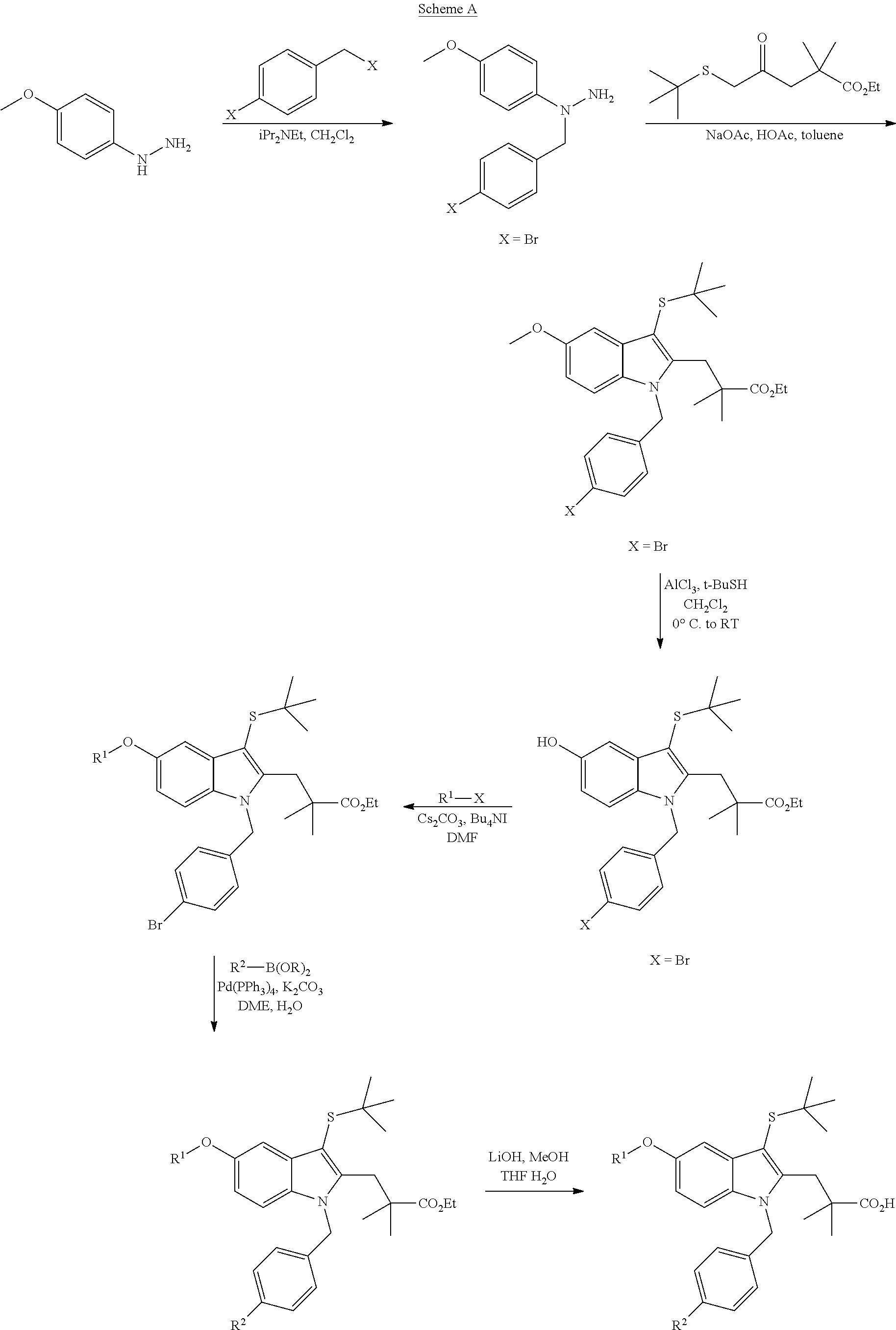

PCT/US2009/44945 describes the Form C polymorph of sodium 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionate and a process for its preparation. The process comprises dissolving 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid ethyl ester in ethanol and tetrahydrofuran, and adding aqueous sodium hydroxide. The mixture is then heated for 16 hours, filtered and then concentrated. The concentrate is then reslurried by adding methyl-tert-butyl ether and heated for 5 hours with stirring. The solids are isolated by filtration and the product dried under vacuum at room temperature for 5 days.

PCT/US2009/44945 also describes a linear process for the preparation of alkyl esters of 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid via the following Scheme B:

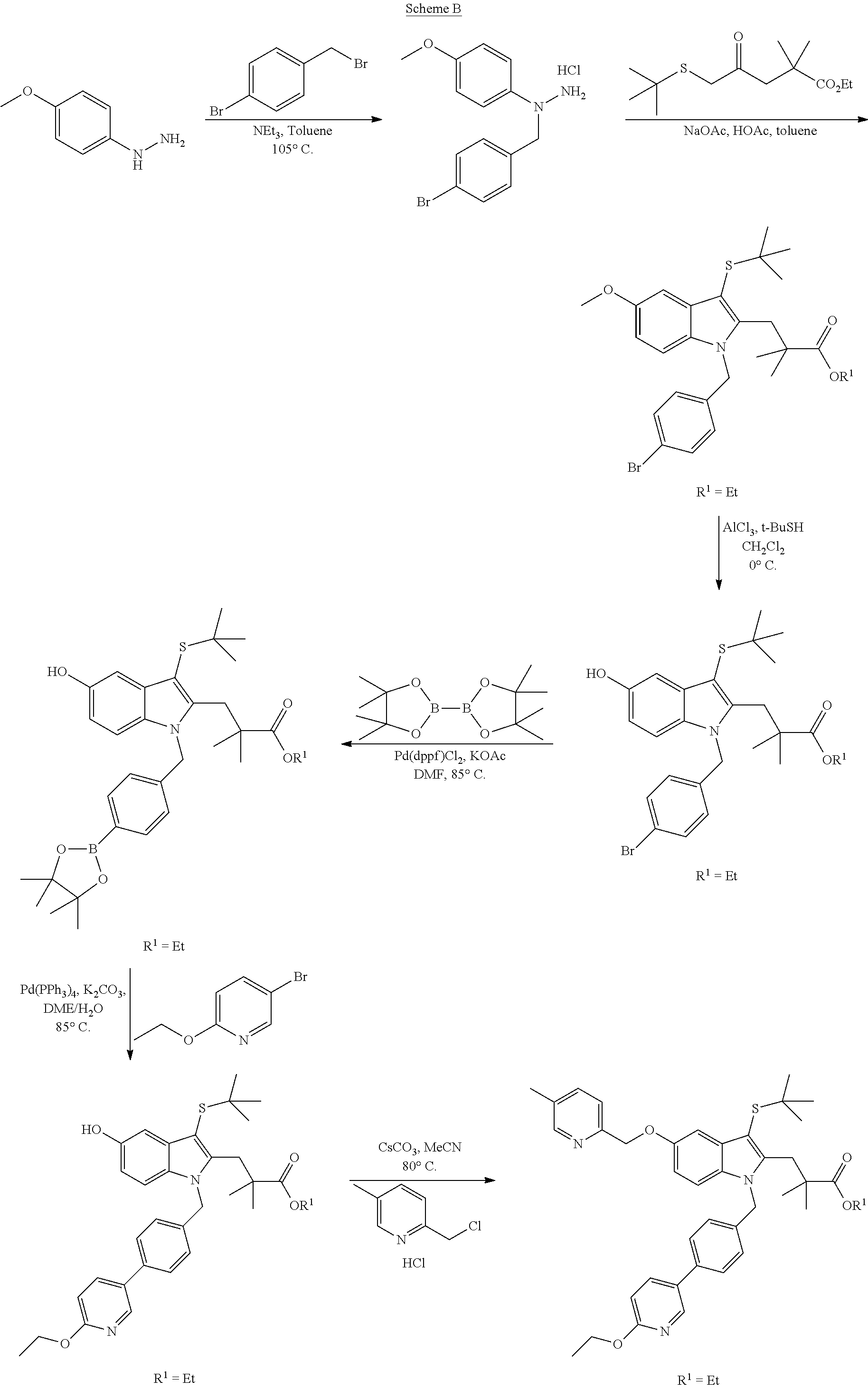

### SUMMARY OF THE INVENTION

Described herein are processes useful for preparing 5-lipoxygenase activating protein (FLAP) inhibitors and their intermediates, for example as shown in Scheme C and Scheme D below. In particular, described herein are processes for preparing 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid, the anhydrous Form C polymorph of sodium 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionate, and intermediates useful in said processes.

Such processes offer advantages over the prior art in that they are convergent rather than linear. Convergent processes may allow for a reduced cycle time, as stages of the reaction scheme may be run in parallel, and an increase in throughput and overall yield. In one comparison, the process of the present invention as shown in Scheme C below, increased the overall chemical yield by a factor of approximately 8, compared to Scheme B of PCT/US2009/44945 beginning with the respective starting materials.

Furthermore, the amount of solvent used in the process of the present invention is reduced compared to Scheme A of WO 2007/056021 and Scheme B of PCT/US2009/44945, thus minimising waste and environmental impact. In particular, the processes of present invention avoid a number of solvents of concern, such as, dichloromethane and acetonitrile, dimethylformamide and 1,2-dimethoxyethane.

The process of the present invention avoids the use of highly undesirable agents such as aluminium chloride, again minimising environmental impact.

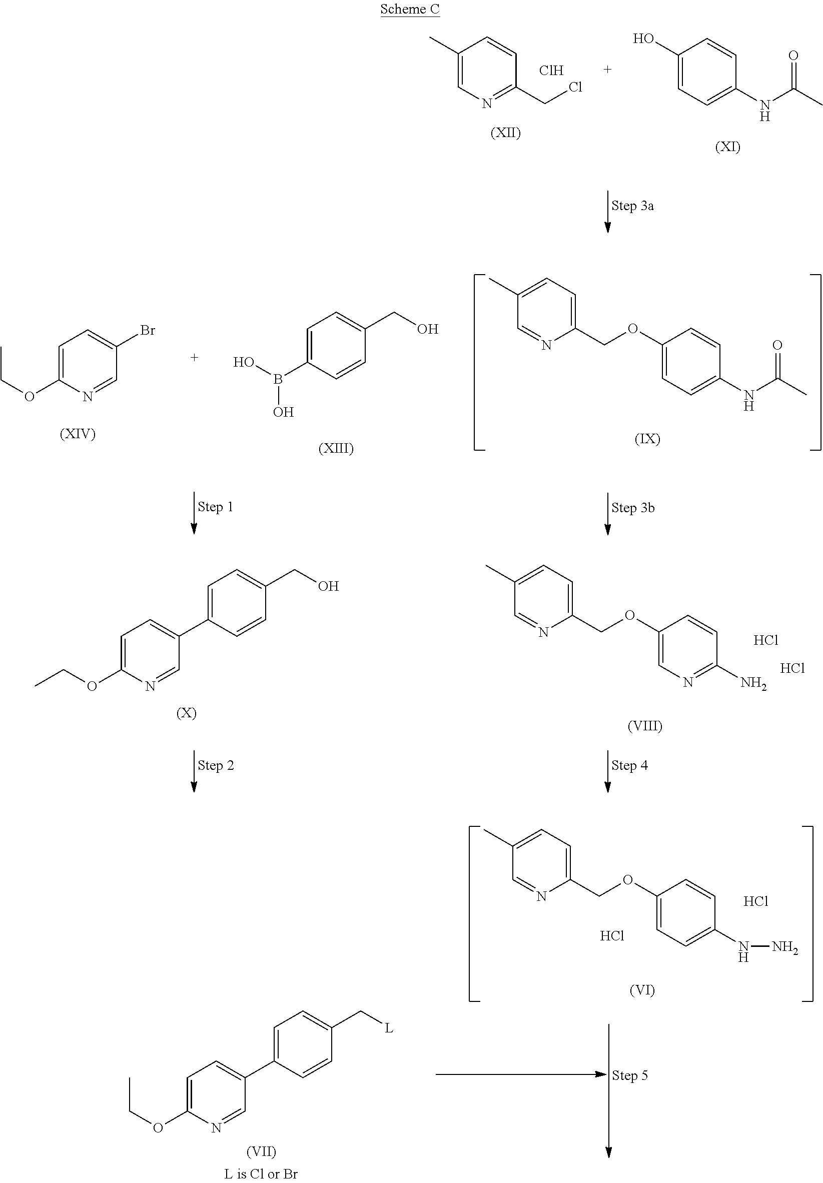

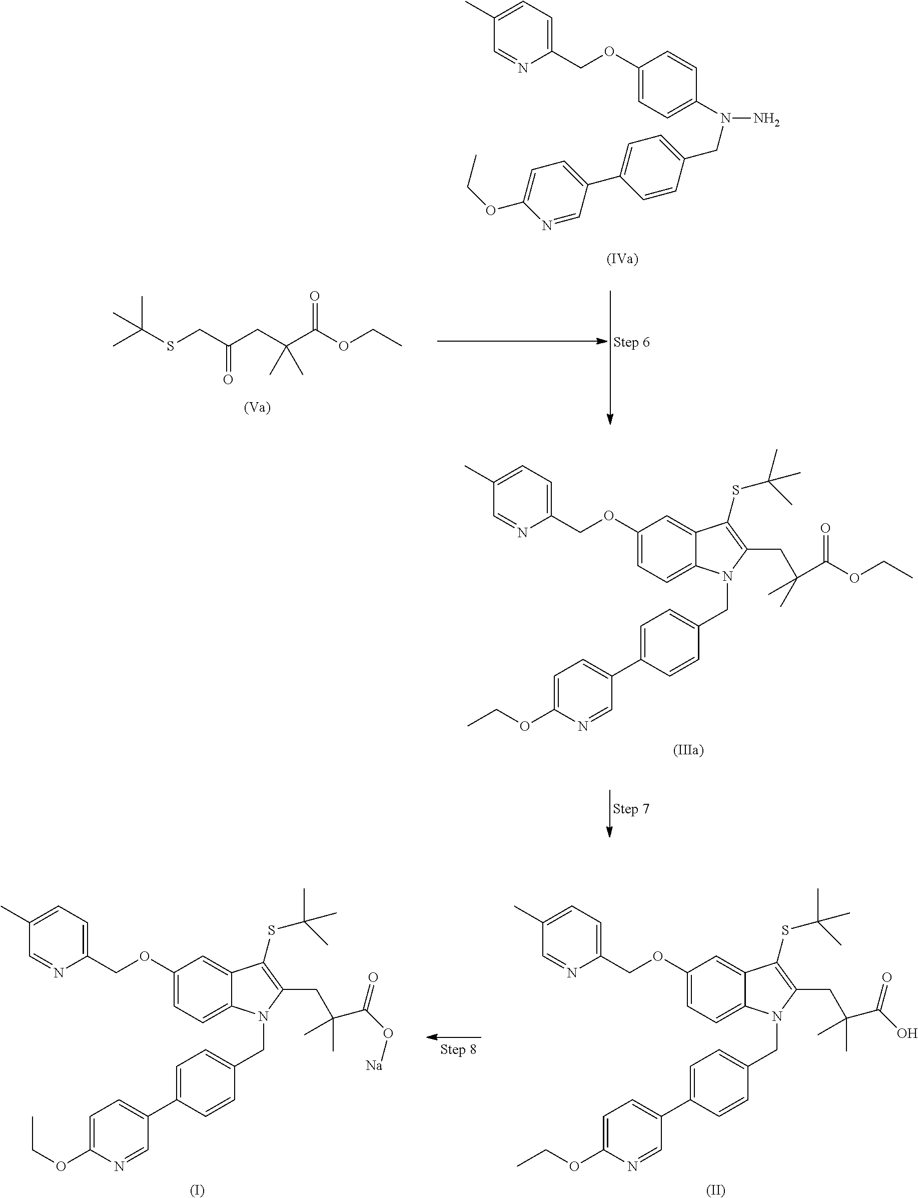

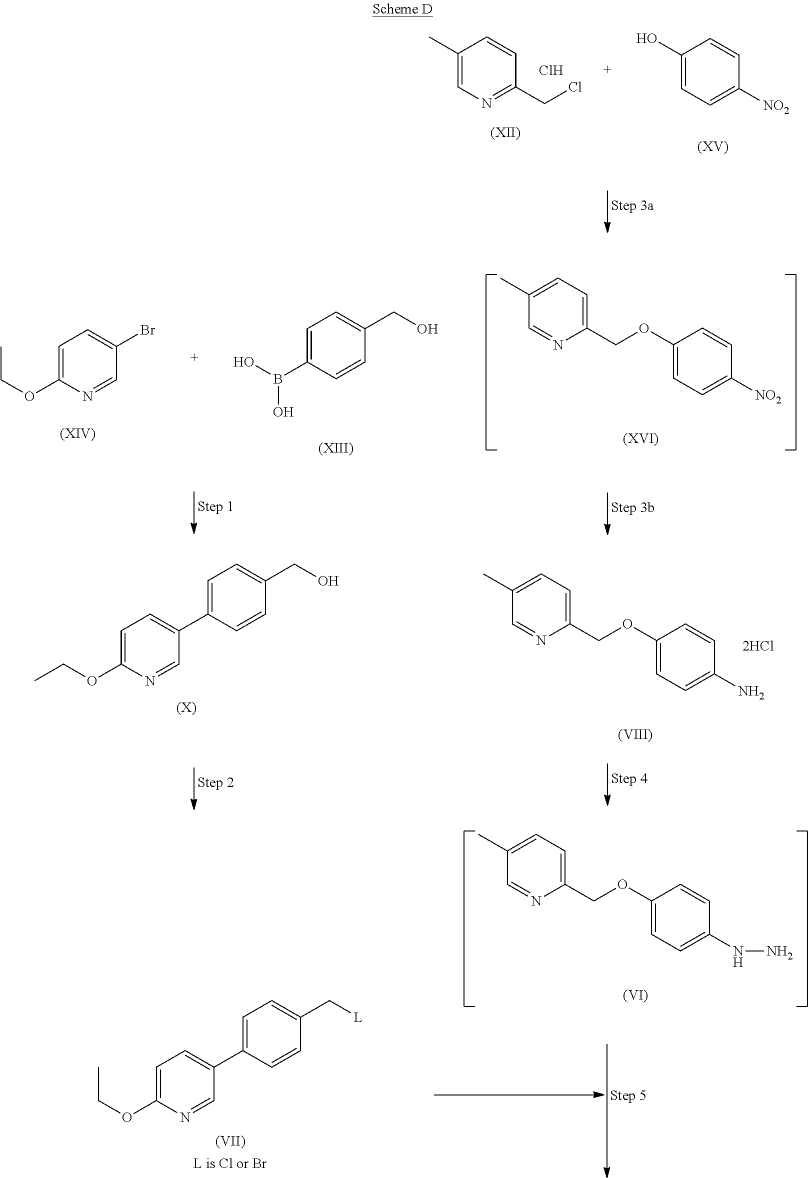

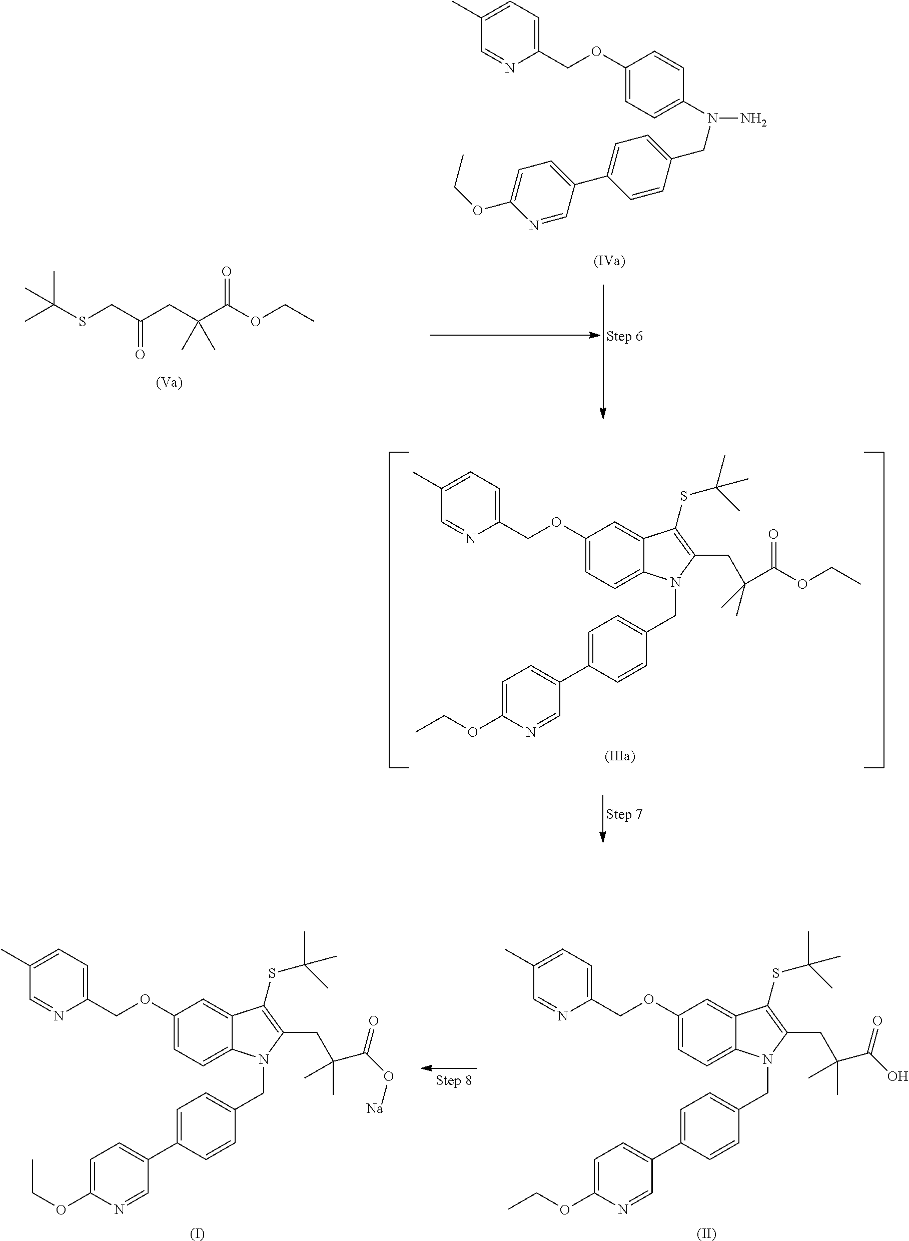

In one aspect of the invention, there is provided a process 1B for preparing the anhydrous Form C polymorph of a compound of formula (I)

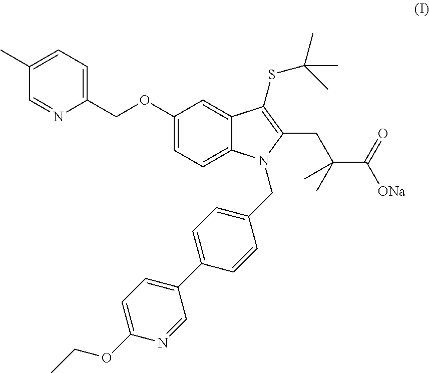

comprising

A) the reaction of a compound of formula (XV)

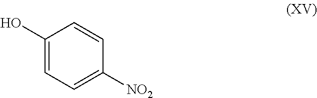

or a salt thereof; with a compound of formula (XII)

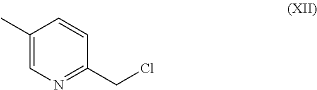

or a salt thereof;
in the presence of a base, and a solvent to produce a compound of formula (XVI)

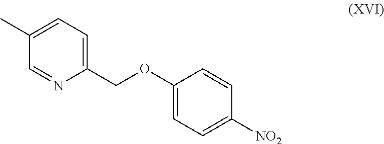

or a salt thereof;

B) followed by the reduction of a compound of formula (XVI) or a salt thereof with hydrogen in the presence of palladium in a solvent, to produce a compound of formula (VIII)

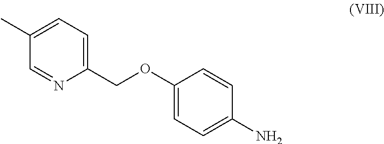

or a salt thereof;

C) followed by the reaction of a compound of formula (VIII) or a salt thereof; with aqueous sodium nitrite in the presence of hydrochloric acid to form the diazonium salt followed by reduction of the diazonium salt to produce a compound of formula (VI)

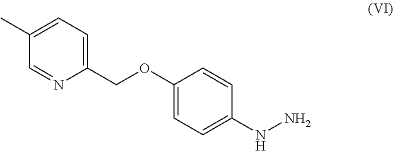

or a salt thereof;

D) the reaction of a compound of formula (XIII)

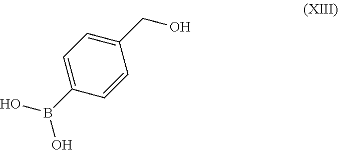

or a salt thereof;
with a compound of formula (XIV)

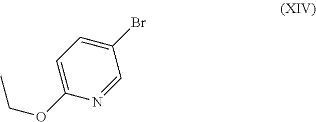

or a salt thereof;
in the presence of a base, aqueous alcoholic solvent and palladium on carbon to produce a compound of formula (X)

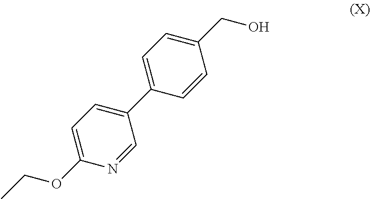

or a salt thereof;

E) followed by the reaction of a compound of formula (X) or a salt thereof; with, when L is bromine, aqueous or anhydrous hydrogen bromide, or where L is chlorine, aqueous or anhydrous hydrogen chloride, cyanuric chloride, thionyl chloride, methane sulfonyl chloride, toluene sulfonyl chloride or phosphoryl chloride to produce a compound of formula (VII)

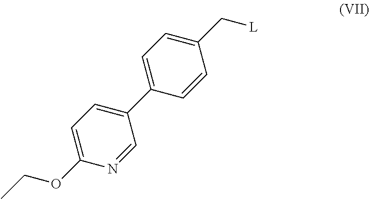

wherein L is chlorine or bromine; or a salt thereof;

F) followed by the step of reacting a compound of formula (VII) or a salt thereof;

wherein L is chlorine or bromine;
with a compound of formula (VI) or a salt thereof;
in the presence of a base and solvent; to produce a compound of formula (IVa)

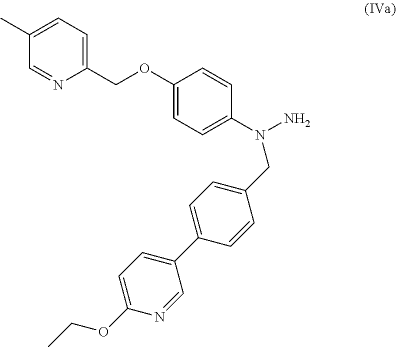

or a salt thereof;

G) followed by the reaction of a compound of formula (IVa) or a salt thereof; with a compound of formula (Va)

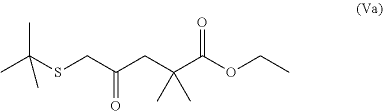

in the presence of an acid and a solvent to produce a compound of formula (IIIa)

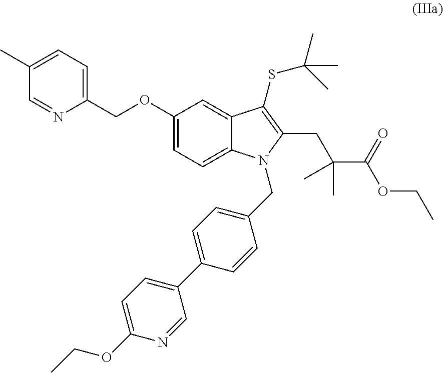

or a salt thereof;

H) followed by the reaction of a compound of formula (IIIa) or a salt thereof with an aqueous solution of a base to produce a compound of formula (II)

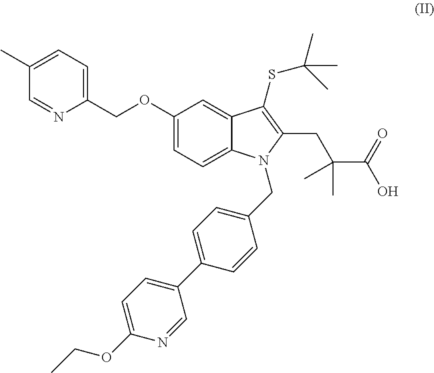

I) followed by
(a) dissolving a compound of formula (II) in methanol and methyl-t-butylether in the presence of solid sodium hydroxide, followed by addition of methyl-t-butylether, wherein the solvent system in the reactant mixture contains 30% or less methanol; or
(b) dissolving a compound of formula (II) in an alcohol which is ethanol or methanol and reacting with aqueous sodium hydroxide, followed by the addition of diisopropylether, wherein the aqueous content of the reaction mixture is ≦5% and the solvent system in the reactant mixture contains 30% or less ethanol or methanol by volume.

In another aspect of the invention, there is provided a process 1 for preparing a compound of formula (ID

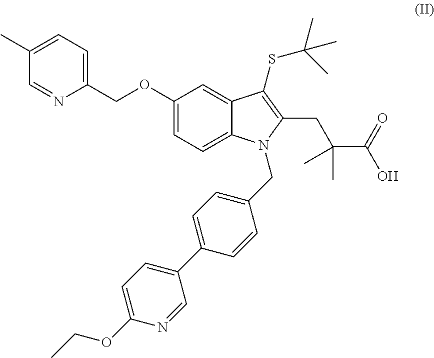

or a salt thereof;
comprising reacting a compound of formula (VII)

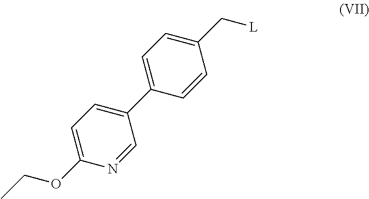

or a salt thereof;
wherein L is a leaving group;
with a compound of formula (VI)

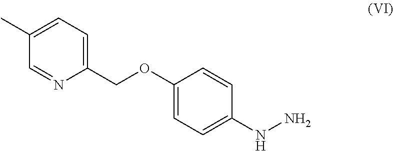

or a salt thereof
in the presence of a base and solvent, and then converting to a compound of formula (II) or a salt thereof.

In another aspect of the invention, there is provided a process 2 for preparing a compound of formula (I)

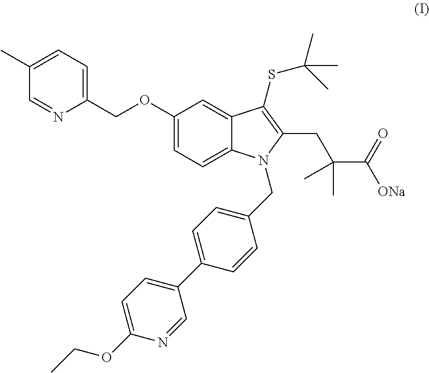

comprising the step of reacting a compound of formula (VII)

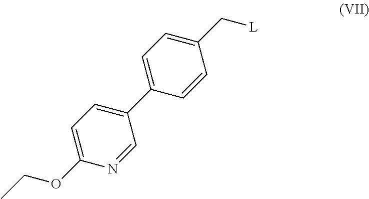

or a salt thereof;
wherein L is a leaving group;
with a compound of formula (VI)

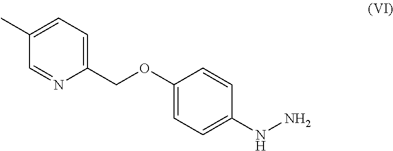

or a salt thereof;
in the presence of a base and solvent, and then converting to a compound of formula (I).

In another aspect of the invention, we have found improved processes for preparing the Form C polymorph of sodium 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionate.

In one embodiment of the invention, there is provided a process 3 for preparing the anhydrous Form C polymorph of a compound of formula (I)

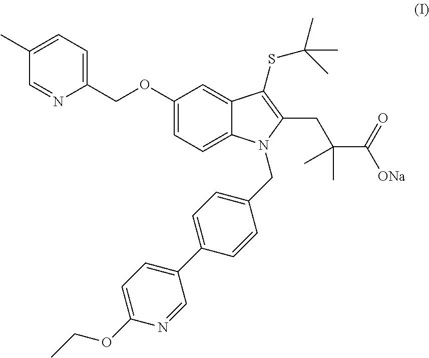

comprising dissolving a compound of formula (II)

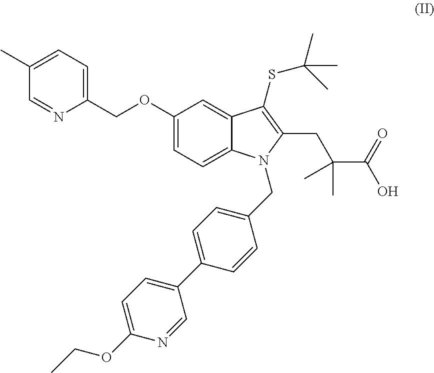

in methanol and methyl-t-butylether in the presence of solid sodium hydroxide, followed by addition of methyl-t-butylether, wherein the solvent system in the reactant mixture contains 30% or less methanol by volume.

In an alternative embodiment of the invention there is provided a process 4 for preparing the anhydrous Form C polymorph of a compound of formula (I)

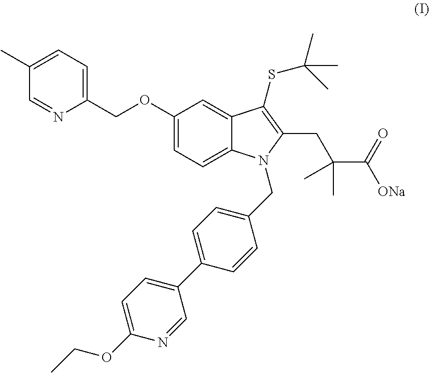

comprising dissolving a compound of formula (II)

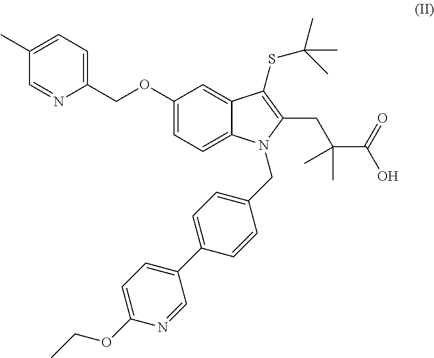

in an alcohol which is methanol or ethanol and reacting with aqueous sodium hydroxide, followed by the addition of diisopropylether, wherein the aqueous content of the reaction mixture is ≦5% and the solvent system in the reactant mixture contains 30% or less methanol or ethanol by volume.

Processes 3 and 4 provide a direct means of crystallisation and avoids having to concentrate the mixture to dryness and then tritarate with methy-t-butylether. Thus the process may allow for greater control and more consistent particle size and physical properties. Furthermore, the use of solid sodium hydroxide in process 3 reduces the amount of water present and makes it easier to control hydrate formation.

In another aspect of the invention, there are provided processes for preparing key intermediates for use in the process for preparing FLAP inhibitors via a Fischer Indole reaction.

In one embodiment, there is provided a process 5 for preparing a compound of formula (III):

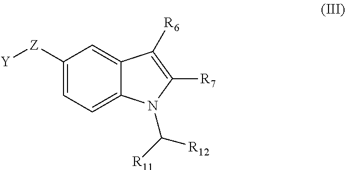

wherein,
Z is selected from —[C(R₁)₂]ₘ[C(R₂)₂]ₙ, —[C(R₂)₂]ₙ[C(R₁)₂]ₘO, —O[C(R₁)₂]ₘ[C(R₂)₂ₙ, or —[C(R₁)₂]ₙOC(R₂)₂]ₙ, wherein each

R₁ is independently H, —CF₃, or —C₁-C₆alkyl or two R₁ on the same carbon may join to form an oxo (═O); and each

R₂ is independently H, —OH, —OMe, —CF₃, or —C₁-C₆alkyl or two R₂ on the same carbon may join to form an oxo (═O);

m is 1 or 2; each

n is independently 0, 1, 2, or 3;

Y is a heteroaryl optionally substituted by halogen, —C₁-C₆alkyl, —C(O)CH₃, —OH, —C₃-C₆cycloalkyl, —C₁-C₆alkoxy, —C₁-C₆fluoroalkyl, —C₁-C₆fluoroalkoxy or —C₁-C₆hydroxyalkyl; R₆ is L₂-R₁₃ wherein

L₂ is a bond, O, S, —S(═O), —S(═O)₂ or —C(═O);

R₁₃ is —C₁-C₆alkyl wherein —C₁-C₆alkyl may be optionally substituted by halogen; R₇ is selected from —C₁-C ₆alkyleneC(O)OC₁₋C₆alkyl, —C₁-C ₆alkyleneC(O)OH and —C₁-C ₆alkyl; R₁₁ is -L₁₀-X-G₆, wherein

L₁₀ is aryl or heteroaryl;

X is a bond, —CH₂— or —NH—;

G₆ is aryl, heteroaryl, cycloalkyl or cycloheteroalkyl optionally substituted by 1 or 2 substituents independently selected from halogen, —OH, —CN, —NH₂, —C₁-C₆alkyl, —C₁-C₆alkoxy, —C₁-C₆fluoroalkyl, —C₁-C₆fluoroalkoxy, —C(O)NH₂ and —NHC(O)CH₃;

R₁₂ is H or —C₁-C₆alkyl; or a
salt thereof;
comprising reacting a compound of formula (IV)

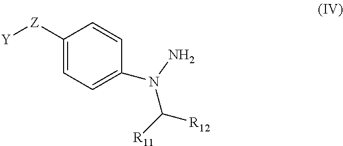

or a salt thereof;
wherein Y, Z, R₁₁ and R₁₂ are as defined for a compound of formula (III) with a compound of formula (V)

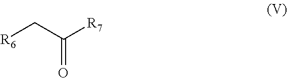

wherein R₆ and R₇ are as defined for the compound of formula (III) in the presence of an acid and solvent.

In another embodiment, there is provided a process 6 for preparing a compound of formula (IIIa)

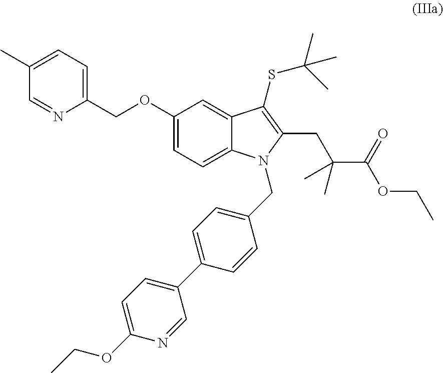

comprising reacting a compound of formula (IVa)

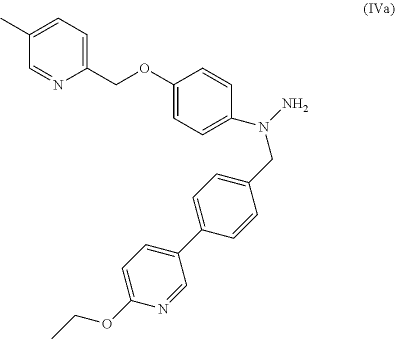

or a salt thereof;
with a compound of formula (Va)

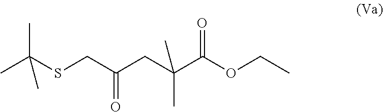

in the presence of an acid and a solvent.

In another aspect of the invention there is provided a process 7 for preparing a compound of formula (II)

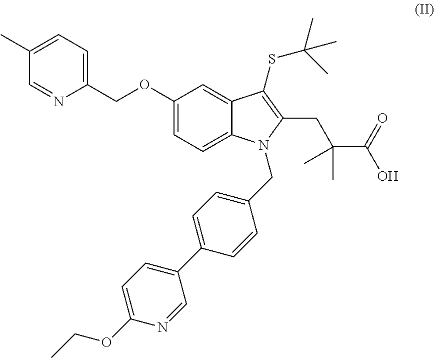

or a salt thereof;
comprising a process for preparing a compound of formula (IIIa) as defined above, and then converting to a compound of formula (II) or a salt thereof.

In a further aspect of the invention there is provided a process 8 for preparing a compound of formula

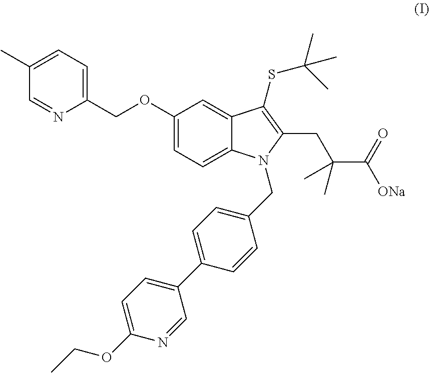

comprising a process for preparing a compound of formula (IIIa) as defined above, and then converting to a compound of formula (I).

### BRIEF DESCRIPTION OF FIGURES

FIG. 1 presents a DSC thermogram of the Form C Polymorph of a Compound of Formula (I) produced via Step 8A (see Examples Section).

FIG. 2 presents an XRPD profile of the Form C Polymorph of a Compound of Formula (I) produced via Step 8A (see Examples Section).

### DETAILED DESCRIPTION OF THE INVENTION

In one aspect of the invention, there is provided a process 1B for preparing the anhydrous Form C polymorph of a compound of formula (I)

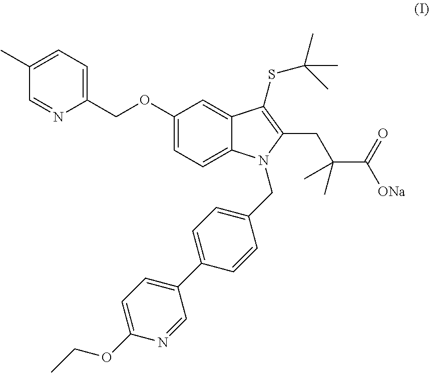

comprising

A) the reaction of a compound of formula (XV)

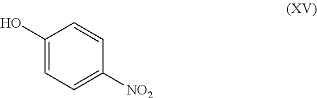

or a salt thereof; with a compound of formula (XII)

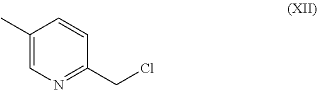

or a salt thereof;
in the presence of a base, and a solvent to produce a compound of formula (XVI)

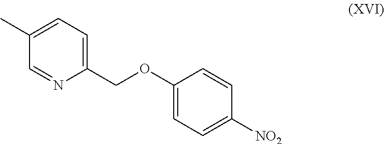

or a salt thereof;

B) followed by the reduction of a compound of formula (XVI) or a salt thereof with hydrogen in the presence of palladium in a solvent, to produce a compound of formula (VIII)

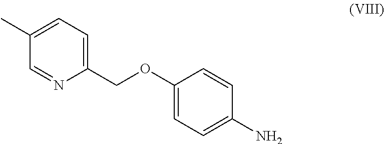

or a salt thereof;

C) followed by the reaction of a compound of formula (VIII) or a salt thereof; with aqueous sodium nitrite in the presence of hydrochloric acid to form the diazonium salt followed by reduction of the diazonium salt to produce a compound of formula (VI)

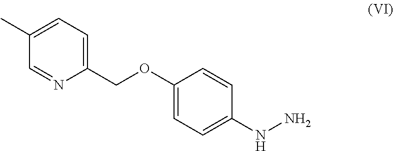

or a salt thereof;

D) the reaction of a compound of formula (XIII)

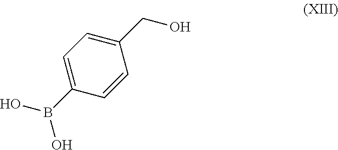

or a salt thereof;
with a compound of formula (XIV)

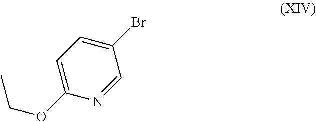

or a salt thereof;
in the presence of a base, aqueous alcoholic solvent and palladium on carbon to produce a compound of formula (X)

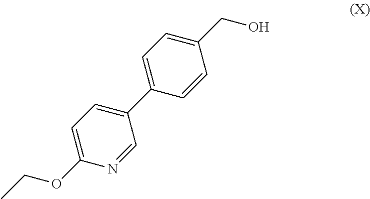

or a salt thereof;

E) followed by the reaction of a compound of formula (X) or a salt thereof; with, when L is bromine, aqueous or anhydrous hydrogen bromide, or where L is chlorine, aqueous or anhydrous hydrogen chloride, cyanuric chloride, thionyl chloride, methane sulfonyl chloride, toluene sulfonyl chloride or phosphoryl chloride to produce a compound of formula (VII)

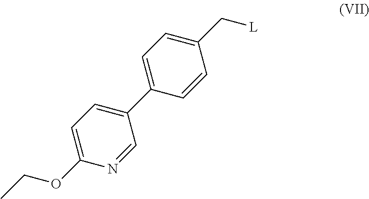

wherein L is chlorine or bromine; or a salt thereof;

F) followed by the step of reacting a compound of formula (VII) or a salt thereof; wherein L is chlorine or bromine;

with a compound of formula (VI) or a salt thereof;
in the presence of a base and solvent; to produce a compound of formula (IVa)

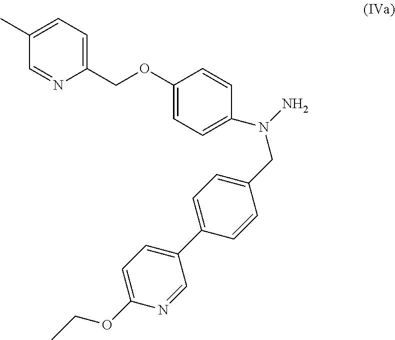

or a salt thereof;

G) followed by the reaction of a compound of formula (IVa) or a salt thereof;

with a compound of formula (Va)

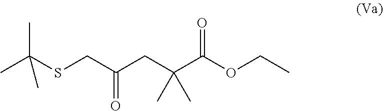

in the presence of an acid and a solvent to produce a compound of formula (IIIa)

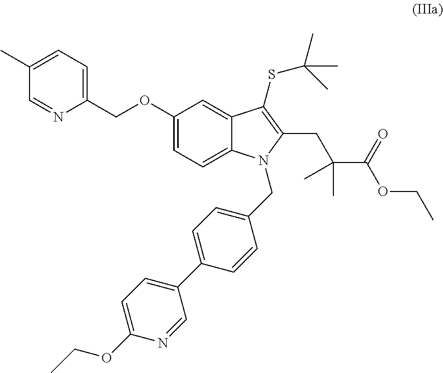

or a salt thereof;

H) followed by the reaction of a compound of formula (IIIa) or a salt thereof with an aqueous solution of a base to produce a compound of formula (II)

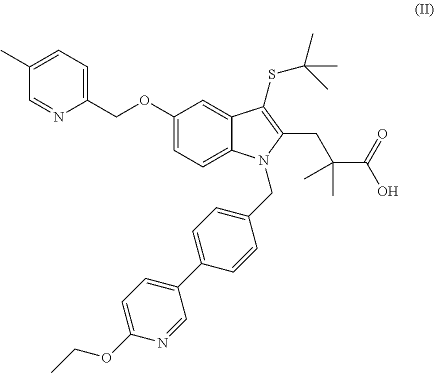

I) followed by
(a) dissolving a compound of formula (II) in methanol and methyl-t-butylether in the presence of solid sodium hydroxide, followed by addition of methyl-t-butylether, wherein the solvent system in the reactant mixture contains 30% or less methanol; or
(b) dissolving a compound of formula (II) in an alcohol which is ethanol or methanol and reacting with aqueous sodium hydroxide, followed by the addition of diisopropylether, wherein the aqueous content of the reaction mixture is ≦5% and the solvent system in the reactant mixture contains 30% or less ethanol or methanol by volume.

In another aspect of the invention, there is provided a process 1 for preparing a compound of formula (II)

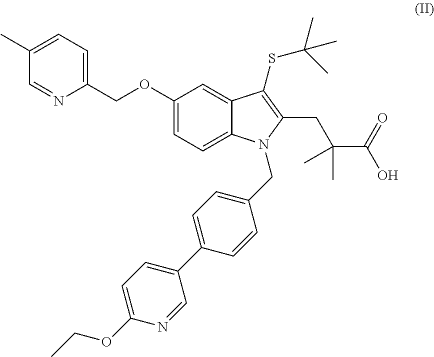

or a salt thereof;
comprising reacting a compound of formula (VII)

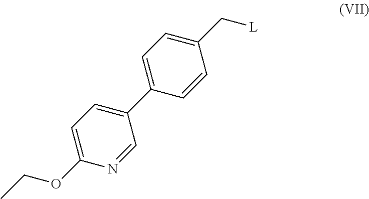

or a salt thereof;
wherein L is a leaving group;
with a compound of formula (VI)

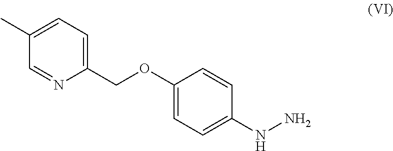

or a salt thereof in the presence of a base and solvent, and then converting to a compound of formula (II) or a salt thereof.

In one embodiment there is provided a process 1 for preparing a compound of formula (II) or a salt thereof. In a further embodiment there is provided a process 1 for preparing a compound of formula (II).

In another aspect of the invention, there is provided a process 2 for preparing a compound of formula (I)

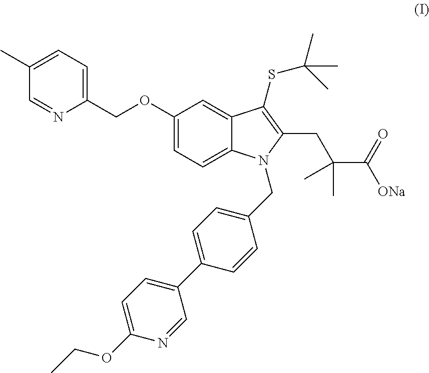

comprising reacting a compound of formula (VII)

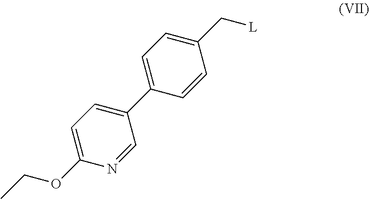

or a salt thereof
wherein L is a leaving group;
with a compound of formula (VI)

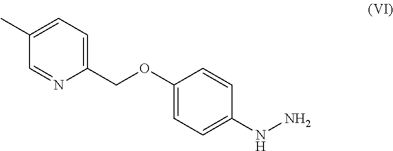

or a salt thereof in the presence of a base and solvent, and then converting to a compound of formula (I).

In one embodiment of process 1 or process 2, L is selected from chlorine and bromine. In another embodiment, L is bromine. In a further embodiment, L is chlorine.

In one embodiment of process 1 or process 2, the base is selected MOH, M₂CO₃ and MHCO₃ wherein M is selected from Li (lithium), Na (sodium), K (potassium) and Cs (caesium); 1,8-diazabicyclo[5.4.0]undec-7-ene; and R′R″R″′N wherein R′, R″ and R″′ are each independently C₁-C₆alkyl. In another embodiment, the base is MOH. In another embodiment, the base is NaOH (sodium hydroxide). In another embodiment the base is KOH (potassium hydroxide). In another embodiment, the base is R′R″R″′N wherein R′, R″ and R″′ are each independently C₁-C₆alkyl. In a further embodiment, the base is R′R″R″′N and R′, R″ and R″′ are each ethyl.

In one embodiment of process 1 or process 2, the base is present to neutralise or part neutralise any acid. In one embodiment the pH of the mixture is ≧4.0. In another embodiment the pH of the mixture is from about 6 to 7.5.

In one embodiment of process 1 or process 2, the reaction is carried out at from about 15° C. to about 21° C. when L is bromine. In another embodiment of process 1 or process 2, the reaction is carried out at from about 40° C. to about 50° C. when L is chlorine.

In one embodiment of process 1 or process 2, the solvent is selected from water, C₁-C₆alcohol, tetrahydrofuran, 2-methyltetrahydrofuran, toluene, dichloromethane and mixtures thereof. In another embodiment, the solvent is selected from C₁-C₆alcohol, tetrahydrofuran, 2-methyltetrahydrofuran, toluene, dichloromethane and mixtures thereof. In another embodiment, the solvent is C₁-C₆alcohol. In another embodiment, the solvent is selected from ethanol, 1-propanol, 2-propanol, 2-butanol, sec-butanol and mixtures thereof. In another embodiment, the solvent is 2-propanol. In another embodiment, the solvent is 2-propanol and water. In a further embodiment, the solvent is tetrahydrofuran.

In one embodiment of process 1 or process 2, the compound of formula (VII) is in the form of a salt or as the free base. In another embodiment the compound of formula (VII) is the free base. In another embodiment the compound of formula (VII) is a salt. In another embodiment the compound of formula (VII) is a salt selected from hydrogen bromide, hydrogen chloride, hydrogen iodide, p-toluenesulfonate, methanesulfonate, trifluoromethanesulfonate and phosphate. In a further embodiment the compound of formula (VII) is a salt selected from hydrogen bromide and hydrogen chloride.

In one embodiment of process 1 or process 2, the compound of formula (VI) is in the form of a salt or as the free base. In another embodiment the compound of formula (VI) is the free base. In another embodiment the compound of formula (VI) is a salt. In a further embodiment the compound of formula (VI) is the dihydrogen chloride salt.

In another aspect of the invention, we have found improved processes for preparing the anhydrous Form C polymorph of a compound of formula (I)

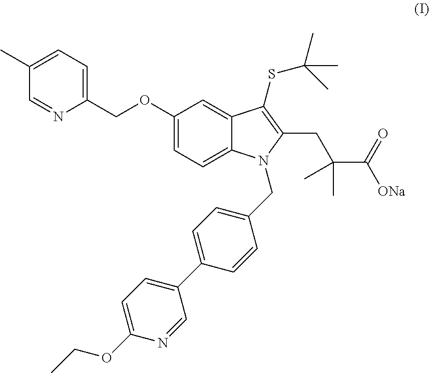

In one embodiment of the invention, there is provided a process 3 for preparing the anhydrous Form C polymorph of a compound of formula (I)

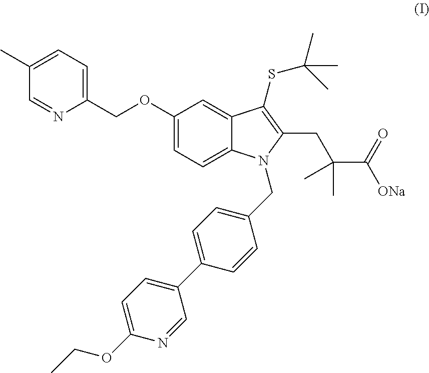

comprising dissolving a compound of formula (II)

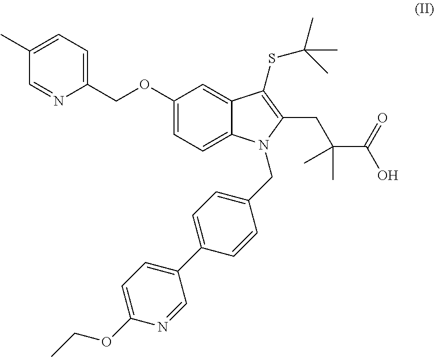

in methanol and methyl-t-butylether in the presence of solid sodium hydroxide, followed by addition of methyl-t-butylether, wherein the solvent system in the reactant mixture contains 30% or less methanol by volume.

In one embodiment the reaction is seeded with the anhydrous Form C polymorph of the compound of formula (I). It should be noted that the anhydrous Form C polymorph of the compound of formula (I) will still be produced without seeding.

Such a process provides a direct means of crystallisation and avoids having to concentrate the mixture to dryness and then tritarate with methy-t-butylether. Thus the process may allow for greater control and more consistent particle size and physical properties. Furthermore, the use of solid sodium hydroxide reduces the amount of water present and makes it easier to control hydrate formation.

In one embodiment, the reaction is carried out at from about 48° C. to about 55° C. By carrying out the reaction at about 48° C. or above the chance of forming alternative polymorphs is significantly reduced. The about 55° C. limit is governed by the solvent boiling point.

In one embodiment, approximately 1.01 equivalents (relative to the compound of formula (II)) of sodium hydroxide is used in the reaction, this prevents the resulting product from being contaminated with excess starting material or sodium hydroxide.

It is possible to recover additional compound of formula (I) from the mother liquor and washes from process 3, by removal of methanol or methanol and methyl-t-butylether by distillation. It may also be possible to perform the same operation using, for example, pervaporation or vapour permeation as an alternative method of methanol removal. It may also be possible to combine the recovery of compound of formula (I) with the recovery of solvent via these latter processes. The additional compound of formula (I) may be the anhydrous Form C Polymorph and/or may require further processing in order to be suitable for clinical use. Such recovery processes should allow for an increase in yield, help reduce cost of goods, increase overall mass productivity and decrease the amount of waste associated with the process.

The recovery of methyl-t-butylether and methanol from a methyl-t-butylether/methanol solvent system may be possible. Such a mixture forms a low boiling azeotrope. Recovery by conventional distillation would require high energy input and would result in losses of methyl-t-butylether product to waste. Alternative technologies such as the use of membranes were investigated together with a hybrid involving distillation and a membrane process. With pervaporation/vapour permeation, liquid mixtures can be separated by selectively evaporating one component from the mixture through a membrane. The membrane only allows the component with the smallest molecular size to be evaporated. The use of a hybrid pervaporation/distillation unit represents the introduction of a low energy technology. Such membranes allow the recovery of methyl-t-butylether and methanol to the required purity (e.g. >99% w/w) and may be purchased from, for example, Sulzer Chemtech GmbH, Friedichsthaler Strasse 19, D-66540 Neunkirchen, Germany. Such solvent recovery would avoid incineration of solvent and hence a reduction in CO₂ emissions from fossil fuel combustion, and a reduction in cost of goods.

In one aspect of the invention, there is provided a process for preparing the anhydrous Form C polymorph of a compound of formula (I)

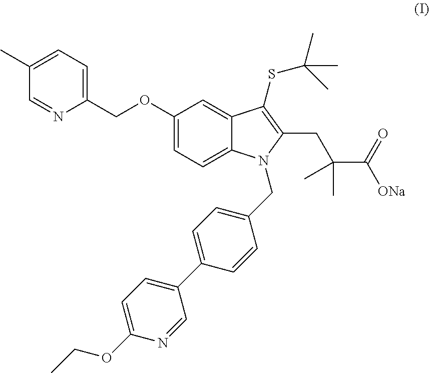

followed by solvent recovery using a pervaporation membrane.

In one embodiment of the invention, there is provided a process 3A for preparing the anhydrous Form C polymorph of a compound of formula (I)

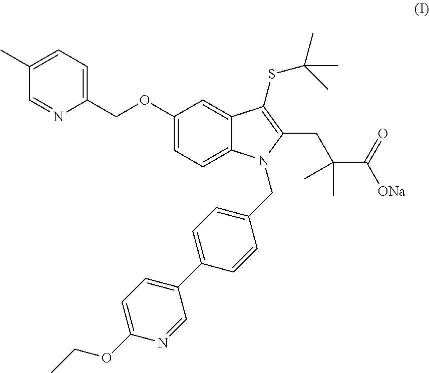

comprising dissolving a compound of formula (II)

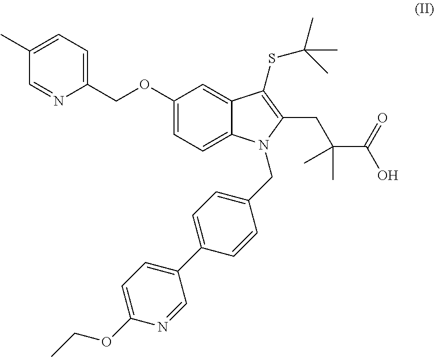

in methanol and methyl-t-butylether in the presence of solid sodium hydroxide, followed by addition of methyl-t-butylether, wherein the solvent system in the reactant mixture contains 30% or less methanol by volume; followed by methanol removal using a pervaporation membrane.

In an alternative embodiment of the invention, there is provided a process 4 for preparing the anhydrous Form C polymorph of a compound of formula (I)

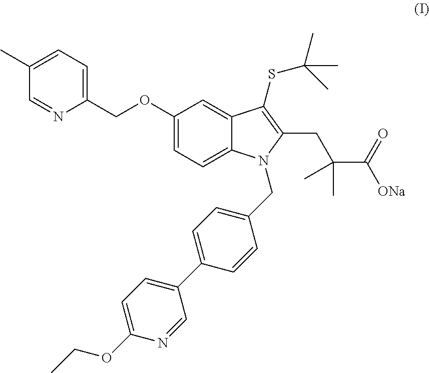

comprising dissolving a compound of formula (II)

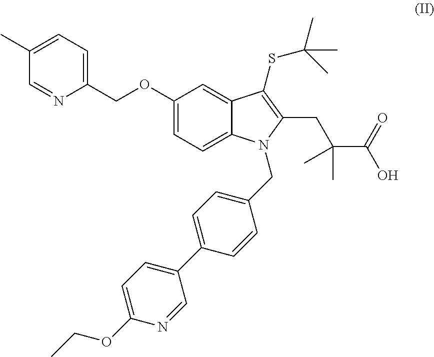

in an alcohol which is ethanol or methanol and reacting with aqueous sodium hydroxide, followed by the addition of diisopropylether, wherein the aqueous content of the reaction mixture is ≦5% and the solvent system in the reactant mixture contains 30% or less ethanol or methanol by volume.

In one embodiment the reaction is seeded with the anhydrous Form C polymorph of the compound of formula (I). It should be noted that the anhydrous Form C polymorph of the compound of formula (I) will still be produced without seeding.

In this embodiment, the aqueous content is preferably kept to a minimum in order to avoid the formation of hydrates, whilst using enough water to ensure the solubility of the compound of formula (II).

In one embodiment, the alcohol is selected from methanol and ethanol. In a further embodiment the alcohol is ethanol.

In one embodiment, the process is conducted at from about 48° C. to about 78° C. By carrying out the reaction at about 48° C. or above the chance of forming alternative polymorphs is significantly reduced. The about 78° C. limit is governed by the solvent boiling point.

In one embodiment, the aqueous content of the reaction mixture is ≦3%. In a further embodiment, the aqueous content of the reaction mixture is ≦2%.

Such a process provides a direct means of crystallisation and avoids having to concentrate the mixture to dryness and then tritarate with methy-t-butylether. Thus the process may allow for a high degree of control and consistent particle size and physical properties.

In one embodiment, the compound of formula (II) may be prepared by ester hydrolysis comprising the reaction of a compound of formula (IIIa)

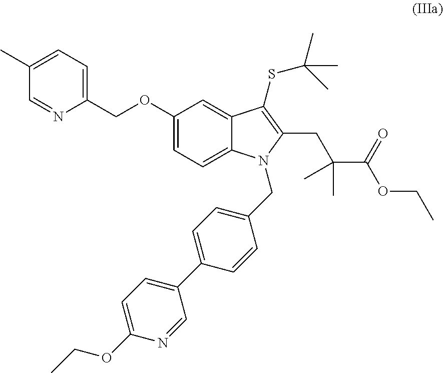

with an aqueous solution of a base.

In one embodiment, the base is selected from MOH wherein M is selected from Li (lithium), Na (sodium), K (potassium) and Cs (caesium); M′(OH)₂ wherein M′ is selected from Ca (calcium) and Ba (barium). In a further embodiment, the base is NaOH (sodium hydroxide).

In one embodiment, the process is carried out in solvent selected from C₁-C₆alcohol, tetrahydrofuran, 2-methyltetrahydrofuran, tetrahydropyran and mixtures thereof. In another embodiment, the process is carried out in a solvent selected from a tetrahydrofuran and ethanol mixture; a methyltetrahydrofuran and methanol mixture; and butanol. In a further embodiment, the process is carried out in a solvent which is a 2-methyltetrahydrofuran and 2-propanol mixture.

In a further aspect of the invention, there is provided a process for preparing key intermediates of formula (III), including compounds of formula (IIIa), as defined above, for use in the process for preparing FLAP inhibitors via a Fischer Indole reaction.

In one embodiment, there is provided a process 5 for preparing a compound of formula (III):

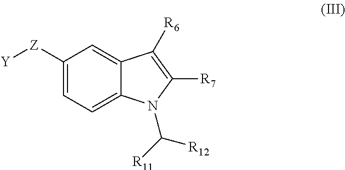

wherein,
Z is selected from —[C(R₁)₂]ₘ[C(R₂)₂]ₙ, —[C(R₂)₂]ₙ[C(R₁)₂]ₘO, —O[C(R₁)₂]ₘ[C(R₂)₂]ₙ, or —[C(R₁)₂]ₙO[C(R₂)₂]ₙ, wherein each

R₁ is independently H, —CF₃, or —C₁-C₆alkyl or two R₁ on the same carbon may join to form an oxo (═O); and each

R₂ is independently H, —OH, —OMe, —CF₃, or —C₁-C₆alkyl or two R₂ on the same carbon may join to form an oxo (═O);

m is 1 or 2; each

n is independently 0, 1, 2, or 3;

Y is a heteroaryl optionally substituted by halogen, —C₁-C₆alkyl, —C(O)CH₃, —OH, —C₃-C₆cycloalkyl, —C₁-C₆alkoxy, —C₁-C₆fluoroalkyl, —C₁-C₆fluoroalkoxy or —C₁-C₆hydroxyalkyl; R₆ is L₂-R₁₃ wherein

L₂ is a bond, O, S, —S(═O), —S(═O)₂ or —C(═O);

R₁₃ is —C₁-C₆alkyl wherein —C₁-C₆alkyl may be optionally substituted by halogen;

R₇ is selected from —C₁-C₆alkyleneC(O)OC₁-C₆alkyl, —C₁-C₆alkyleneC(O)OH and —C₁-C₆alkyl;
R₁₁ is -L₁₀-X-G₆, wherein

L₁₀ is aryl or heteroaryl;

X is a bond, —CH₂— or —NH—;

G₆ is aryl, heteroaryl, cycloalkyl or cycloheteroalkyl optionally substituted by 1 or 2 substituents independently selected from halogen, —OH, —CN, —NH₂, —C₁-C₆alkyl, —C₁- C₆alkoxy, —C₁-C₆fluoroalkyl, —C₁-C₆fluoroalkoxy, —C(O)NH₂ and —NHC(O)CH₃;

R₁₂ is H or —C₁-C₆alkyl; or a
salt thereof;
comprising the reaction of a compound of formula (IV)

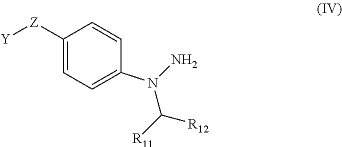

or a salt thereof;
wherein Y, Z, R₁₁ and R₁₂ are as defined for a compound of formula (III) with a compound of formula (V)

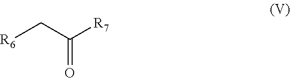

wherein R₆ and R₇ are as defined for the compound of formula (III) in the presence of an acid and solvent.

In one embodiment, Z is —O[C(R₁)₂]ₘ[C(R₂)₂]ₙ, R₁ is H, m is 1 and n is 0.

In one embodiment, Y is heteroaryl optionally substituted by —C₁-C₆alkyl. In another embodiment, Y is pyridinyl optionally substituted by —C₁-C₆alkyl. In another embodiment, Y is pyridinyl optionally substituted by methyl. In a further embodiment, Y is 5-methyl-pyridinyl.

In one embodiment, R₁₃ is —C₁-C₆alkyl and L₂ is S, —S(═O) or —S(═O)₂. In a further embodiment, R₁₃ is tert-butyl and L₂ is S.

In one embodiment, R₇ is C₁-C₆alkyleneC(═O)OC₁-C₆alkyl. In another embodiment, R₇ is C₄alkyleneC(═O)OC₁₋₆alkyl. In another embodiment, R₇ is —CH₂C(CH₃)₂C(═O)OC₁-C₆alkyl. In another embodiment, R₇ is —CH₂C(CH₃)₂C(═O)OCH₃. In a further embodiment, R₇ is —CH₂C(CH₃)₂C(═O)OCH₂CH₃.

In one embodiment, L₁₀ is aryl, X is a bond and G₆ is heteroaryl. In another embodiment, L₁₀ is aryl, X is a bond and G₆ is heteroaryl substituted by —OH or —C₁-C₆alkoxy. In another embodiment, L₁₀ is aryl, X is a bond and G₆ is heteroaryl substituted by —OCH₃ or —OCH₂CH₃. In another embodiment, L₁₀ is phenyl, X is a bond and G₆ is heteroaryl substituted by —OCH₃ or —OCH₂CH₃. In another embodiment, L₁₀ is phenyl, X is a bond and G₆ is pyridinyl substituted by —OCH₃ or —OCH₂CH₃. In a further embodiment, L₁₀ is phenyl, X is a bond and G₆ is pyridinyl substituted by —OCH₂CH₃.

In another embodiment, there is provided a process 6 or preparing a compound of formula (IIIa)

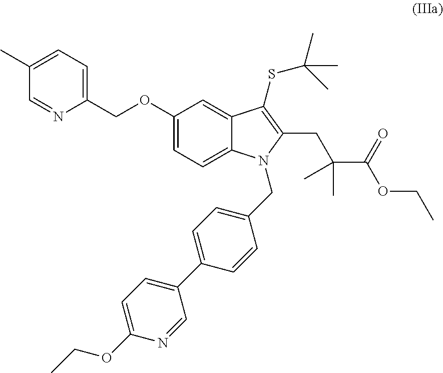

comprising the reaction of a compound of formula (IVa)

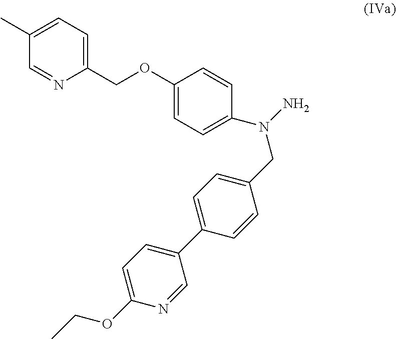

or a salt thereof;
with a compound of formula (Va)

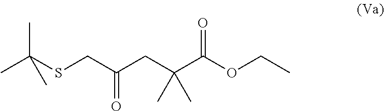

in the presence of an acid and a solvent.

In one embodiment of process 5 or 6, the compound of formula (IV) or (IVa) is in the form of a salt or as the free base. In another embodiment, the compound of formula (IV) or (IVa) is the free base. In another embodiment, the compound of formula (IV) or (IVa) is a salt. In another embodiment, the compound of formula (IV) or (IVa) is a salt selected from hydrogen bromide, hydrogen chloride, hydrogen iodide, p-toluenesulfonate, methanesulfonate, trifluoromethanesulfonate, phosphate, citrate, tartrate, formate, acetate and propionate. In a further embodiment, the compound of formula (IV) or (IVa) is salt selected from hydrogen bromide and hydrogen chloride.

In one embodiment of process 5 or 6, the solvent is selected from a C₁-C₆alcohol, tetrahydrofuran, 2-methyltetrahydrofuran, water and mixtures thereof. In another embodiment the solvent is selected from a C₁-C₆alcohol, tetrahydrofuran, 2-methyltetrahydrofuran and mixtures thereof. In another embodiment, the solvent is a C₁-C₆alcohol selected from ethanol, 2-propanol and mixtures thereof. In a further embodiment the solvent is a mixture of 2-methyltetrahydrofuran, 2-propanol and water.

In one embodiment of process 5 or 6, the acid is a carboxylic acid. In another embodiment, the carboxylic acid is selected from the group consisting of isobutyric acid, citric acid, tartaric acid, acetic acid, propanoic acid, butanoic acid, dibenzoyl tartaric acid (for example, dibenzoyl tartaric acid monohydrate or dibenzoyl tartaric acid anhydrous), ditoluoyl tartaric acid, malic acid, maleic acid, benzoic acid, 3-phenyl acetic acid, triphenylacetic acid, phtalic acid, 2-hydroxyphenylacetic acid, anthracene-9-carboxylic acid, methoxyacetic acid, tartronic acid, glutaric acid, oxalic acid, trichloroacetic acid, camphoric acid, ethylhexanoic acid, napthylacetic acid and mixtures thereof. In another embodiment, the carboxylic acid is selected from the group consisting of isobutyric acid, citric acid, tartaric acid, acetic acid, propanoic acid, butanoic acid, dibenzoyl tartaric acid (for example, dibenzoyl tartaric acid monohydrate or dibenzoyl tartaric acid anhydrous), ditoluoyl tartaric acid, malic acid, benzoic acid, 3-phenyl acetic acid, triphenylacetic acid, phtalic acid, 2-hydroxyphenylacetic acid, anthracene-9-carboxylic acid, methoxyacetic acid, tartronic acid, glutaric acid and mixtures thereof. In another embodiment, the carboxylic acid is selected from isobutyric acid, citric acid, tartaric acid, acetic acid, propanoic acid, butanoic acid, dibenzoyl tartaric acid (for example, dibenzoyl tartaric acid monohydrate), ditoluoyl tartaric acid and mixtures thereof. In a further embodiment, the acid is a carboxylic acid selected from dibenzoyl tartaric acid (for example, dibenzoyl tartaric acid monohydrate) and isobutyric acid.

In one embodiment of process 5 or 6, the acid is a mixture of two or more acids. In another embodiment the acid is dibenzoyl tartaric acid in mixture with a co-acid selected from citric acid, maleic acid, oxalic acid, trichloroacetic acid, sodium hydrogen sulphate, camphoric acid, phosphoric acid, potassium dihydrogen phosphate, ethylhexanoic acid, isobutyric acid and napthylacetic acid. In another embodiment the acid is dibenzoyl tartaric in mixture with a co-acid selected from citric acid, trichloroacetic acid, sodium hydrogen sulphate, isobutyric acid and napthylacetic acid. In a further embodiment the acid is dibenzoyl tartaric in mixture with citric acid.

In one embodiment of process 5 or 6, the reaction is carried out at from about 5° C. to about 70° C. In another embodiment, the reaction is carried out from about 30° C. to about 60° C. In a further embodiment, the reaction is carried out at from about 20° C. to about 50° C.

It may be possible to recover the acid (e.g. dibenzoyl tartaric acid) or acids (e.g. dibenzoyl tartaric acid and citric acid) by partial removal of residual solvent (e.g. 2-methyltetrahydrofuran) followed by acidification with an acid, such as hydrochloric acid. It may also be possible to extract the acid (e.g. dibenzoyl tartaric acid) or acids (e.g. dibenzoyl tartaric acid and citric acid) into a solvent (e.g. 2-methyltetrahydrofuran) at acidic pH and recycle into another reaction directly, or by crystallising from this solvent (e.g. toluene or benzene) and then re-using.

In another aspect of the invention there is provided a process 7 for preparing a compound of formula (II)

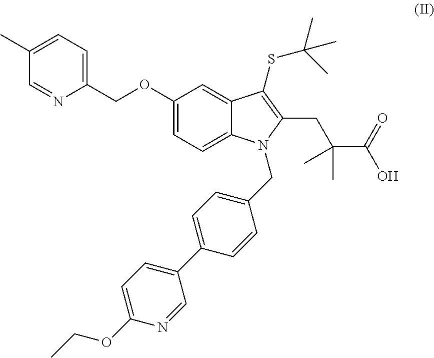

or a salt thereof;
comprising a process for preparing a compound of formula (IIIa) as defined above, and then converting to a compound of formula (II) or a salt thereof.

In one embodiment there is provided a process 7 for preparing a compound of formula (II) or a salt thereof. In a further embodiment there is provided a process 7 for preparing a compound of formula (II).

In one aspect of the invention process 7 is telescoped, wherein the compound of formula (IIIa) is not isolated.

In one embodiment of the invention there is provided a telescoped process 7A for preparing a compound of formula (II)

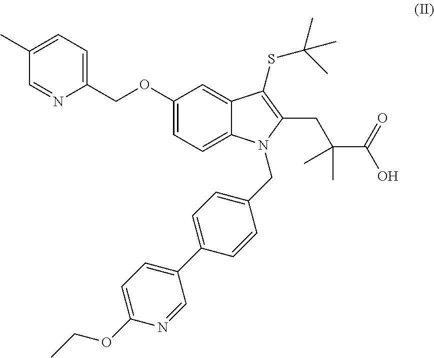

or a salt thereof;
comprising a process for preparing a compound of formula (IIIa) as defined above, followed by ester hydrolysis with a base, in the presence of a C₁-C₆alcohol and a tetrahydrofuran as solvent, and then converting to a compound of formula (II) or a salt thereof.

In one embodiment of the invention there is provided a telescoped process 8A for preparing a compound of formula (I)

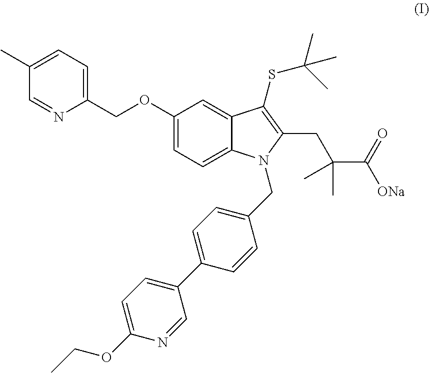

comprising a process for preparing a compound of formula (IIIa) as defined above, followed by ester hydrolysis with a base, in the presence of a C₁-C₆alcohol and a tetrahydrofuran as solvent, and then converting to a compound of formula (I).

In one embodiment of process 7A or process 8A, the reaction is carried out at from about 5° C. to about 70° C. In a further embodiment, the reaction is carried out at from about 30° C. to about 55° C.

In one embodiment, the base is selected from MOH wherein M is selected from Li (lithium), Na (sodium), K (potassium) and Cs (caesium); M′(OH)₂ wherein M′ is selected from Ca (calcium) and Ba (barium). In a further embodiment, the base is NaOH (sodium hydroxide).

In one embodiment the solvent is a mixture of a C₁-C₆alcohol and a tetrahydrofuran. In another embodiment the solvent is a mixture of a C₁-C₆alcohol and 2-methyltetrahydrofuran. In a further embodiment the solvent is a mixture of 2-propanol and 2-methyltetrahydrofuran.

2-Methyltetrahydrofuran is known to be a ‘green’ alternative to tetrahydrofuran. Unlike tetrahydrofuran, 2-methyltetrahydrofuran is obtained from renewable sources such as agricultural by-products. Reduced miscibility with water when compared with tetrahydrofuran is also an advantage when considering solvent recovery opportunities.

It may be possible to recover the 2-methyltetrahydrofuran and 2-propanol solvent. A standard distillation would offer efficient separation but the use of a membrane separation as described above for Process 3, may offer an even more efficient recovery. Such solvent recovery would avoid incineration of solvent and hence a reduction in CO₂ emissions from fossil fuel combustion, and a reduction in cost of goods.

In a further aspect of the invention there is provided a process 8 for preparing a compound of formula (I)

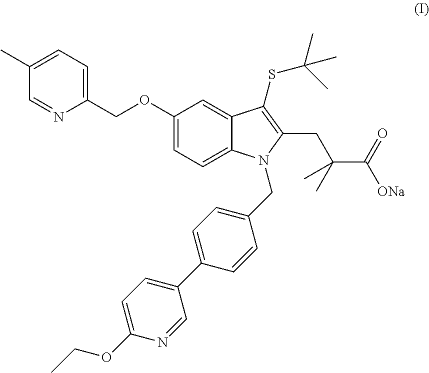

comprising a process for preparing a compound of formula (IIIa) as defined above, and then converting to a compound of formula (I).

Compounds of formula (V) and (Va) may be prepared using methods similar to those described in U.S. Pat. No. 5,288,743. Alternatively the compound of formula (Va) is commercially available and may be purchased from, for example, Aurora Screening Library.

Compounds of formula (IV) and (IVa) may be prepared using methods similar to those described in UK Patent Application No. GB 2 265 621A.

Alternatively, the compound of formula (IVa) may be prepared by the reaction of a compound of formula (VII)

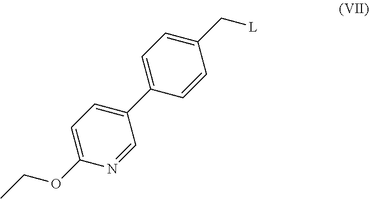

or a salt thereof;
wherein L is a leaving group;
with a compound of formula (VI)

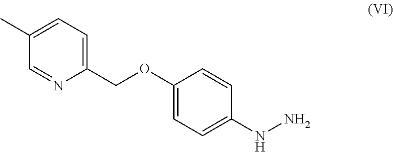

or a salt thereof;
in the presence of a base and a suitable solvent.

In one embodiment, L is selected from chlorine and bromine. In another embodiment, L is bromine. In a further embodiment, L is chlorine.

In one embodiment, the base is selected MOH, M₂CO₃ and MHCO₃ wherein M is selected from Li (lithium), Na (sodium), K (potassium) and Cs (caesium); 1,8-diazabicyclo[5.4.0]undec-7-ene; and R′R″R″′N wherein R′, R″ and R″′ are each independently C₁C₆alkyl. In another embodiment, the base is MOH. In another embodiment the base is NaOH (sodium hydroxide). In another embodiment the base is KOH (potassium hydroxide). In another embodiment, the base is R′R″R″′N wherein R′, R″ and R″′ are each independently C₁-C ₆alkyl. In a further embodiment, the base is R′R″R″′N and R′, R″ and R″′ are each ethyl.

In one embodiment, the base is present to neutralise or part neutralise any acid. In one embodiment the pH of the mixture is ≧4.0. In another embodiment the pH of the mixture is from about 6 to 7.5.

In one embodiment, the reaction is carried out at from about 15° C. to about 21° C. when L is bromine. In another embodiment, the reaction is carried out at from about 40° C. to about 50° C. when L is chlorine.

In one embodiment, there is provided a process for preparing a compound of formula (IVa) wherein the solvent is selected from water, C₁-C₆alcohol, tetrahydrofuran, 2-methyltetrahydrofuran, toluene, dichloromethane and mixtures thereof. In another embodiment, the solvent is C₁-C₆alcohol. In another embodiment, the solvent is selected from C₁-C₆alcohol, tetrahydrofuran, 2-methyltetrahydrofuran, toluene, dichloromethane and mixtures thereof. In another embodiment, the solvent is C₁-C₆alcohol. In another embodiment, the solvent is selected from ethanol, 1-propanol, 2-propanol, 2-butanol, sec-butanol and mixtures thereof. In another embodiment, the solvent is 2-propanol. In another embodiment, the solvent is 2-propanol and water. In another embodiment the solvent is water. In a further embodiment, the solvent is tetrahydrofuran.

In one embodiment, the compound of formula (VII) is in the form of a salt or as the free base. In another embodiment, the compound of formula (VII) is the free base. In another embodiment the compound of formula (VII) is a salt. In another embodiment, the compound of formula (VII) is a salt selected from hydrogen bromide, hydrogen chloride, hydrogen iodide, p-toluenesulfonate, methanesulfonate, trifluoromethanesulfonate and phosphate. In a further embodiment, the compound of formula (VII) is a salt selected from hydrogen bromide and hydrogen chloride.

In one embodiment, the compound of formula (VI) is in the form of a salt or as the free base. In another embodiment, the compound of formula (VI) is the free base. In another embodiment, the compound of formula (VI) is a salt. In a further embodiment, the compound of formula (VI) is the dihydrogen chloride salt.

The compound of formula (VI) may be prepared by the reaction of a compound of formula (VIII)

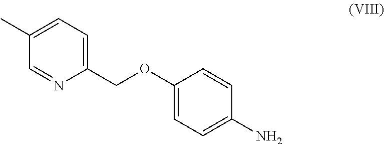

or a salt thereof;
with aqueous sodium nitrite in the presence of hydrochloric acid to form the diazonium salt followed by reduction of the diazonium salt. In one embodiment, the diazonium salt is reduced with an agent selected from ascorbic acid, sodium sulphite, sodium metabisulfite and sodium hydrosulfite. In another embodiment, the diazonium salt is reduced with sodium hydrosulfite

In one embodiment, the compound of formula (VIII) is in the form of a salt or as the free base. In another embodiment, the compound of formula (VIII) is the free base. In another embodiment, the compound of formula (VIII) is a salt. In another embodiment, the compound of formula (VIII) is a salt selected from hydrogen bromide, hydrogen chloride, hydrogen iodide, p-toluenesulfonate, methanesulfonate, trifluoromethanesulfonate, phosphate, citrate, tartrate, formate, acetate and propionate. In a further embodiment, the compound of formula (VIII) is salt selected from hydrogen bromide and hydrogen chloride.

In one embodiment, the process by which the diazonium salt is formed is carried out at from about 0° C. to about 5° C.

In one embodiment the addition of sodium hydrosulfite is carried out at <10° C.

In one aspect of the invention the process for preparing a compound of formula (IV) and the process for preparing a compound of formula (VI) are telescoped, wherein the compound of formula (VI) is not isolated.

In one embodiment of the invention there is provided a telescoped process 1A for preparing a compound of formula (II)

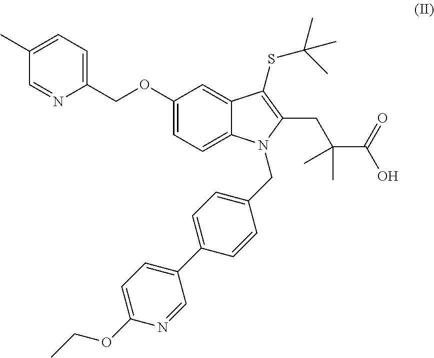

or a salt thereof;
comprising a process for preparing a compound of formula (VI) as defined above, followed by a process for preparing a compound of formula (IVa) as defined above, wherein the compound of formula (VI) is not isolated, and then converting to a compound of formula (II).

In one embodiment of the invention there is provided a telescoped process 2A for preparing a compound of formula (I)

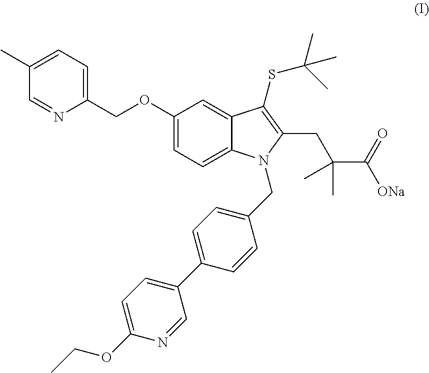

comprising a process for preparing a compound of formula (VI) as defined above, followed by a process for preparing a compound of formula (IVa) as defined above, wherein the compound of formula (VI) is not isolated, and then converting to a compound of formula (I).

The compound of formula (VIII) may be prepared by the reaction of a compound of formula (IX)

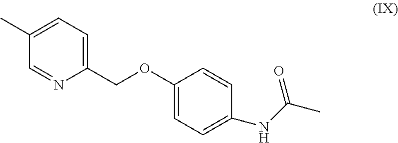

or a salt thereof; with sodium hydroxide in an alcoholic solvent, such as ethanol. In one embodiment the mixture is heated under reflux. The hydrogen chloride salt may then be made by addition of hydrogen chloride in a non-aqueous solvent such as an alcohol, for example, 2-propanol.

The compound of formula (IX) may be prepared by the reaction of a compound of formula (XI)

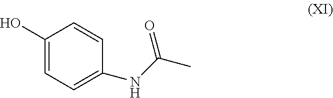

or a salt thereof; with a compound of formula (XII)

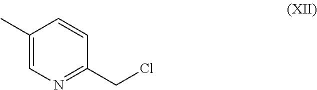

or a salt thereof;
in the presence of a base, and a solvent. In one embodiment, the base is potassium carbonate. In one embodiment, the solvent is ethanol. In one embodiment, the reaction is heated under reflux.

Alternatively, the compound of formula (VIII) may be prepared by the reduction of a compound of formula (XVI)

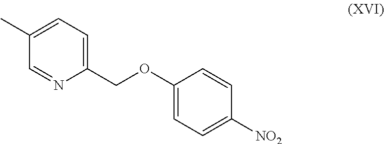

or a salt thereof; with hydrogen in the presence of palladium in a solvent, such as tetrahydrofuran. The hydrogen chloride salt may then be made by addition of hydrogen chloride in a non-aqueous solvent such as an alcohol, for example, 2-propanol.

The compound of formula (XVI) may be prepared by the reaction of a compound of formula (XV)

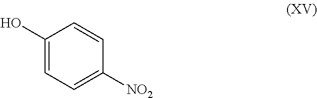

or a salt thereof; with a compound of formula (XII)

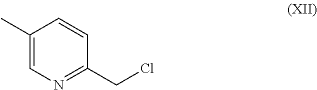

or a salt thereof;
in the presence of a base, and a solvent. In one embodiment, the base is potassium carbonate. In one embodiment, the solvent is dimethylsulfoxide. In one embodiment, the reaction is heated at from 60 to 70° C.

In one embodiment, the compound of formula (XII) is in the form of the hydrochloride salt. The compound of formula (XI) is commercially available and may be purchased from, for example, Aldrich, Fischer Scientific and Univar Limited.

The compound of formula (XV) is commercially available and may be purchased from, for example, Aldrich.

The compound of formula (XII) is commercially available and may be purchased from, for example, Anichem.

In one embodiment, the compound of formula (VII) is prepared via a nucleophilic substitution reaction comprising the reaction of a compound of formula (X)

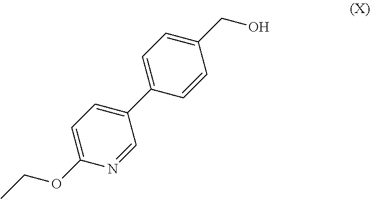

or a salt thereof;
with, when L is bromine, aqueous or anhydrous hydrogen bromide, or where L is chlorine, aqueous or anhydrous hydrogen chloride, cyanuric chloride, thionyl chloride or phosphoryl chloride. In another embodiment the compound of formula (VII) is prepared via a nucleophilic substitution reaction comprising the reaction of a compound of formula (X) or a salt thereof; with aqueous hydrogen bromide (wherein L is bromine) or hydrogen chloride (wherein L is chlorine). In a further embodiment the compound of formula (VII) is prepared via a nucleophilic substitution reaction comprising the reaction of a compound of formula (X) or a salt thereof; with cyanuric chloride (wherein L is chlorine).

In one embodiment, the compound of formula (X) is in the form of a salt or as the free base. In another embodiment, the compound of formula (X) is the free base. In another embodiment, the compound of formula (X) is a salt. In another embodiment, the compound of formula (X) is a salt selected from hydrogen bromide, hydrogen chloride, hydrogen iodide, p-toluenesulfonate, methanesulfonate, trifluoromethanesulfonate and phosphate. In a further embodiment, the compound of formula (X) is a salt selected from hydrogen bromide and hydrogen chloride.

When L is bromine, the process may be carried out at from about 44° C. to about 50° C. When L is chlorine, the chlorinating agent is added at 5 20° C. and the mixture then heated at from about 20° C. to about 35° C.

Alternatively the compound of formula (VII) may be prepared via a nucleophilic substitution reaction comprising the reaction of a compound of formula (X)

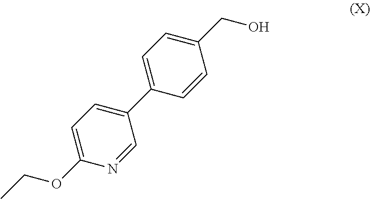

or a salt thereof;
with acetic acid and hydrogen bromide.

In one embodiment, the compound of formula (X) is in the form of a salt or as the free base. In another embodiment, the compound of formula (X) is the free base. In another embodiment, the compound of formula (X) is a salt. In another embodiment, the compound of formula (X) is a salt selected from hydrogen bromide, hydrogen chloride, hydrogen iodide, p-toluenesulfonate, methanesulfonate, trifluoromethanesulfonate and phosphate. In a further embodiment, the compound of formula (X) is a salt selected from hydrogen bromide and hydrogen chloride.

The process may be carried out at from about 44° C. to about 50° C.

The compound of formula (X) may be prepared by a Suzuki cross-coupling reaction comprising the reaction of a compound of formula (XIII)

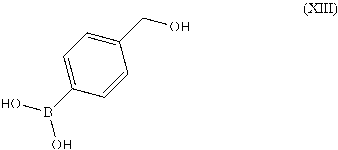

or a salt thereof;
with a compound of formula (XIV)

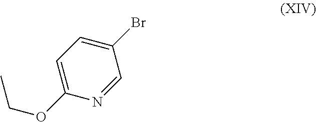

or a salt thereof;
in the presence of a base, aqueous alcoholic solvent and palladium on carbon. In one embodiment the mixture is heated under reflux. In another embodiment, the compound of formula (X) may be prepared by any suitable cross-coupling reaction known to one skilled in the art using appropriate starting materials for example, Kumada-Corriu, Suzuki-Miyaura, Negishi and Stille,

In one embodiment the reaction is seeded with the compound of formula (X). It should be noted that the compound of formula (X) will still be produced without seeding.

In one embodiment, the base is selected from sodium carbonate, sodium hydroxide and potassium carbonate. In a further embodiment, the base is sodium carbonate.

In one embodiment, the aqueous alcohol solvent is selected from methanol, ethanol and propanol. In a further embodiment, the aqueous alcohol solvent is ethanol.

The compound of formula (XIII) is commercially available and may be purchased from, for example, Archimica.

The compound of formula (XIV) is commercially available and may be purchased from, for example, Aldrich and Manchester Organics.

The term “aryl” refers to a C₅-C₁₀ aromatic group which has at least one ring having a conjugated pi electron system and includes both monocyclic or fused-ring polycyclic (i.e., rings which share adjacent pairs of carbon atoms) groups. Examples include phenyl and naphthalene.

The term “alkylene” refers to a divalent C₁-C₆ straight or branched hydrocarbon chain.

The term “alkyl” as used herein as a group or a part of a group refers to a straight or branched hydrocarbon chain containing the specified number of carbon atoms. For example, C₁-C₆alkyl means a straight or branched alkyl containing at least 1, and at most 6, carbon atoms. Examples of “alkyl” as used herein include, but are not limited to, methyl, ethyl, n-propyl, n-butyl, n-pentyl, isobutyl, isopropyl, t-butyl and hexyl.

The term “alkoxy” as used herein as a group or a part of a group refers to a —O(alkyl) group, where “alkyl” is as defined herein.

The term “alcohol” as used herein refers to an alkyl group substituted by a hydroxyl (-OH) group, where “alkyl” is as defined herein. Examples of “alcohol” as used herein include, but are not limited to, methanol, ethanol, propanol and butanol.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Illustrative examples of cycloalkyl groups include the following moieties:

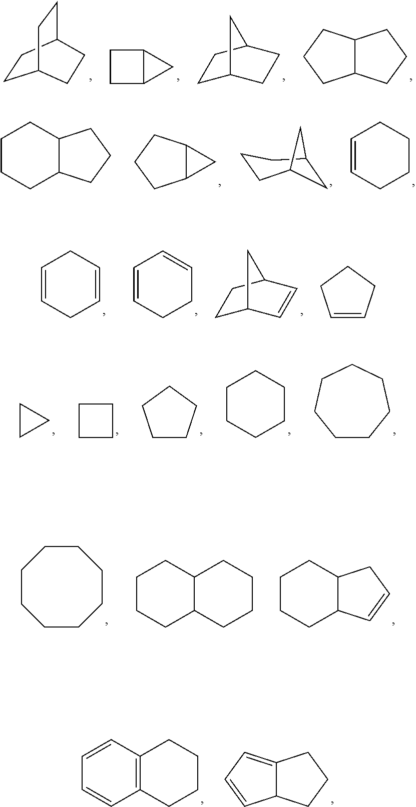

and the like.

The term “cycloheteroalkyl” refers to a C₅-C₆ cycloalkyl group that includes one or more ring heteroatoms selected from nitrogen, oxygen and sulfur. Examples of cycloheteroalkyl groups include tetrahydropyran, tetrahydrofuran, tetrahydrothiophene, piperidine, piperazine, morpholine, 1,4-dioxane, thiomorpholine, 1,4-oxathiane and 1,4-dithane.

The term “halo” or, alternatively, “halogen” means fluoro, chloro, bromo or iodo.

The term “heteroaryl” refers to an aryl or biaryl group that includes one or more ring heteroatoms selected from nitrogen, oxygen and sulfur. An N-containing “heteroaryl” moiety refers to an aromatic group in which at least one of the skeletal atoms of the ring is a nitrogen atom. Examples of heteroaryl groups include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Illustrative examples of heteroaryl groups include the following moieties:

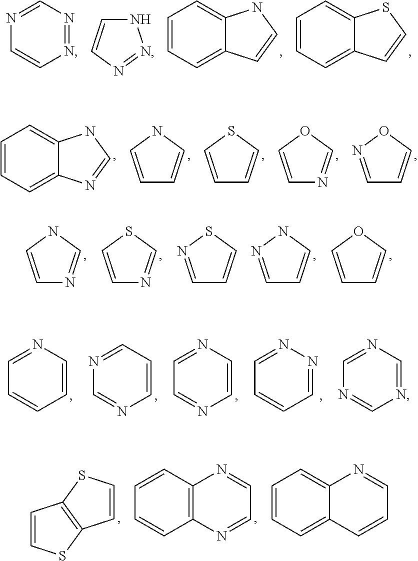

and the like.

### EXAMPLES

### Abbreviations

TBME methyl-tert-butylether
DMSO dimethylsulphoxide
min minutes
NMP N-methyl pyrrolidine
h hours
HPLC high performance liquid chromatography
IPA isopropyl alcohol
2-MeTHF 2-methyltetrahydrofuran
THF tetrahydrofuran
DSC differential scanning calorimetry
XRPD X-ray powder diffraction
   When the term “degassed” is used, this refers to cycles of vacuum/nitrogen purging, with the number of cycles depicted in parentheses.

### Step 1: 5-[4-(Hydroxymethyl)phenyl]-2-(ethyloxy)pyridine

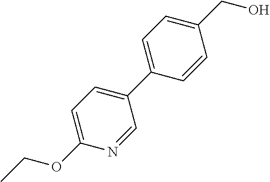

A suspension of 4-(hydroxymethyl)phenyl]boronic acid (14 kg), 5-bromo-2-(ethyloxy)pyridine (19.6 kg), sodium carbonate (11.4 kg) in ethanol (169.4L) and water (49.4L) was stirred under vacuum and then purged with nitrogen twice. A suspension of 10% palladium on carbon (50% wet, 4.6 kg) was added followed by water (7 L), and the suspension was degassed (3×) under nitrogen. The reaction mixture was heated to 63±3° C. and then heated to reflux and stirred for 5 h. The catalyst was filtered off at 57-63° C., and the cake washed with ethanol (28 L). The reaction was concentrated to ca.140 L by atmospheric distillation, cooled to 57±3° C. and water (28 L) added, maintaining >54° C. The reaction was cooled to 53±3° C. and seeded with 5-[4-(hydroxymethyl)phenyl]-2-(ethyloxy)pyridine (70 g) as a slurry in ethanol/water (1:1, 200 mL). After 2 h 10 min water (14 L) was added, maintaining the temperature at 53±3° C. and then cooled to 2±3° C. over about 4.5 hours followed by a 0.5h age. The product was isolated by filtration, washed with ethanol/water (1:1, 140 L) at 2±3° C., followed by water (3×93 L) and dried at 40-50° C. under vacuum to give the title product (19.0 kg, 90% th) as a white solid.

¹H NMR (400 MHz, CHLOROFORM-D) δppm 8.19 (1H, d, J=2.4 Hz); 7.71 (1H, dd, J=8.6, 2.7 Hz); 7.41 (2H, d, J=8.2 Hz); 7.37 (2H, d, J=8.2 Hz); 6.75 (1H, d, J=8.8 Hz); 4.68 (2H, d, J=5.6 Hz); 4.36 (2H, q, J=7.1 Hz); 3.44 (1H, t, J=5.9 Hz); 1.40 (3H, t, J=7.1 Hz).

### Step 2: 5-[4-(Bromomethyl)phenyl]-2-(ethyloxy)pyridine

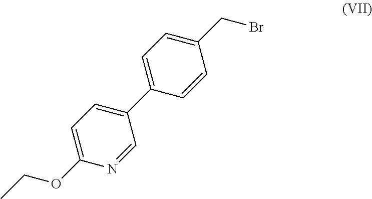

5[4-(hydroxymethyl)phenyl]-2-(ethyloxy)pyridine (47 kg) was stirred and heated to 47±3° C. in hydrogen bromide (48 wt % aq., 709 kg). After about 7 h at this temperature the reaction was cooled to 20±3° C. over 2 h, water (470 L) was then added and the mixture stirred for 1 h. The product was isolated by filtration and the slurry was washed with water (472 kg), aqueous sodium bicarbonate (23.5 kg in 706 kg water) followed by a displacement wash of water (475 kg). The white solid was dried at 30±5° C. under vacuum to give the title product (58.15 kg, 97%) as a white solid.

¹H NMR (400 MHz, DMSO-D6) δppm 8.49 (1H, d, J=2.4 Hz); 8.01 (1H, dd, J=8.7, 2.6 Hz); 7.66 (2H, d, J=8.3 Hz); 7.54 (2H, d, J=8.3 Hz); 6.89 (1H, d, J=8.8 Hz); 4.77 (2H, s); 4.36 (2H, q, J=7.0 Hz); 1.35 (3H, t, J=7.1 Hz).

### Step 2A: 5-[4-(bromomethyl)phenyl]-2-(ethyloxy)pyridine hydrobromide

All weights, volumes and equivalents are relative to 5-[4-(hydroxymethyl)phenyl]-2-(ethyloxy)pyridine. 5[4-(hydroxymethyl)phenyl]-2-(ethyloxy)pyridine (0.910 kg) was heated to 45±3° C. in glacial acetic acid (2.5 vol, 2.28 L). 33wt % hydrogen bromide in acetic acid (2.4 vol, 2.18 L) was added maintaining the temperature below 55° C. After 4 h at 45±3° C., diisopropyl ether (3.0 vol, 2.70 L) was added and the mixture aged 30 min. Diisopropylether (7.0 vol, 6.37 L) was added then the slurry was cooled to 3±3° C. and stirred for 1 h. The product was isolated by filtration and washed three times with diisopropyl ether (6vol, 5.46 L). The material was dried at 40±5° C. under vacuum to give the title product (1.43 kg, 96%) as a white solid.

¹H NMR (400 MHz, CHLOROFORM-D) δppm 8.65 (1H, d, J=2.20 Hz); 8.39 (1H, dd, J=9.05, 2.45 Hz); 7.52-7.58 (4H, m); 7.29 (1H, d, J=9.05 Hz); 4.83 (2H, q, J=6.93 Hz); 4.54 (2H, s); 1.61 (3H, t, J=7.09 Hz).

### Step 2B: 5-[4-(chloromethvl)phenyl]-2-(ethyloxy)pyridine

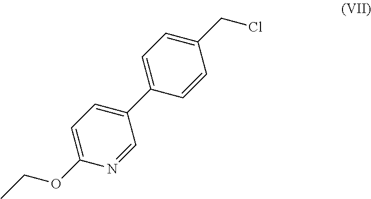

5-[4-(Hydroxymethyl)phenyl]-2-(ethyloxy)pyridine (1.0 kg., 1.00 eq.) was dissolved in tetrahydrofuran (2.5 L) and dimethyl sulfoxide (0.5 L) under an atmosphere of nitrogen. The mixture was cooled to 0±3° C. and cyanuric chloride (320 g, 0.40 eq.) was added maintaining the internal temperature below 20° C. The mixture was heated to 23±3° C. and stirred until there was less than 2.0% a/a 5[4-(hydroxymethyl)phenyl]-2-(ethyloxy)pyridine by HPLC analysis. The slurry was filtered and the cake washed with tetrahydrofuran (0.5 L) and iso-propanol (5.0 L). Water (14 L) was added to the combined filtrate, maintaining the temperature below 35° C. The resulting slurry was cooled to 23±3° C., aged and filtered. The cake was washed with water (3×10 L), pulled dry and dried at 45±5° C. in a vacuum oven to give the title compound (959 g, 89%) as a white powder.

¹H NMR (400 MHz, DMSO-d₆) d ppm 8.48 (1 H, d, J=2.45 Hz) 8.00 (1 H, dd, J=8.68, 2.57 Hz) 7.67 (2 H, d, J=8.07 Hz) 7.52 (2 H, d, J=8.07 Hz) 6.88 (1 H, d, J=8.56 Hz) 4.81 (2 H, s) 4.36 (2 H, q, J=7.09 Hz) 1.34 (3 H, t, J=6.97 Hz).

### Step 3: 4-{[(5-Methylpyridin-2-yl)methyl]oxy}aniline dihydrochloride

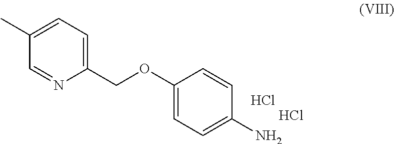

N-(4-Hydroxyphenyl)acetamide (25.0 kg) and potassium carbonate (50.0 kg) were mixed in ethanol (187.5 L) at 22±3° C. and 2-(chloromethyl)-5-methylpyridine hydrochloride (32.5 kg) was added portionwise at 22±3° C. The mixture was then heated to reflux for 15 h. The reaction was then cooled to 57±3° C. and water (162.5 L) added maintaining this temperature. The organic and aqueous phases were allowed to separate and the lower aqueous layer was removed. The organic layer was then washed with aqueous potassium carbonate (20% w/v, 114 kg) at 57±3° C. Sodium hydroxide (50% w/v, 57.8 kg) was then added together with ethanol (12.5 L) and the reaction stirred at reflux for about 38 h. The reaction was cooled to 57±3° C. and the lower aqueous phase was removed. The organic layer was concentrated to ˜125 L by atmospheric distillation, 2-butanol (250 L) was then added and the concentration repeated. The reaction was then cooled to 22±3° C., further 2-butanol (125 L) was added and the mixture washed with water (75 L) at 50±3° C., followed by aqueous sodium chloride (5% w/w, 78 kg) at 50±3° C. The reaction was concentrated to 125 L by atmospheric distillation, further 2-butanol (125 L) was then added and the concentration repeated. 2-Propanol (150 L) was then added followed by hydrogen chloride (5M-6M in 2-propanol, 89.5 kg) over 2 hours at 76±3° C. The resulting slurry was then cooled to 22±3° C. over about 3.5 h, aged for about 40 min and the product isolated by filtration, washed with 2-propanol (2×200 L) follow by TBME (200 L) and dried at 40-50° C. under vacuum to give the title product (40.25 kg, 85% th).

¹H NMR (400 MHz, DMSO-D6) δppm 8.74 (1H, s); 8.28 (1H, dd, J=8.2, 1.3 Hz); 7.91 (1H, d, J=8.1 Hz); 7.38-7.42 (2H, m); 7.17-7.21 (2H, m); 5.46 (2H, s); 2.45 (3H, s).

### Step 3A: Alternative Synthesis of 4-{[(5-methylpyridin-2-yl)methyl]oxy}aniline dihydrochloride

4-Nitrophenol (43 kg) and potassium carbonate (150 kg) were slurried in dimethylsulfoxide (217 L) under an atmosphere of nitrogen. 2-(Chloromethyl)-5-methylpyridine hydrochloride (58 kg) was added to the slurry and the mixture heated to 65±3° C. and stirred for 3 h. Water (866 L) was added maintaining the temperature above 55° C., the slurry was aged for 1 h, cooled to 20±3° C. over 2 h and aged for 1 h. The slurry was filtered and the solid washed with water (433 L), followed by aqueous iso-propanol (50% v/v, 2×433L) and water (2×346 L). The cake was blown with 2 barg nitrogen for 6 h to yield 5-methyl-2-[(4-nitrophenoxy)methyl]pyridine.

The 5-methyl-2-[(4-nitrophenoxy)methyl]pyridine (86.2 kg)* was dissolved in tetrahydrofuran (700 L) and 10% palladium on carbon (50% aqueous paste, 1.7kg) was added. The vessel was purged with nitrogen before three vacuum/nitrogen cycles, followed by three vacuum/hydrogen cycles. An atmosphere of 2 barg hydrogen was placed on the vessel and the mixture stirred vigorously at 23±3° C. for 8 h and the mixture filtered to remove palladium. The filter was washed with tetrahydrofuran (350 L) followed by iso-propanol (350 L) and the combined filtrate distilled to 420 L. iso-Propanol (700 L) was added, the solution distilled to 420L vol and iso-propanol (980 L) added. Hydrochloric acid in iso-propanol (5.5 mol dm⁻³, 156 L) was added over 1 h, maintaining the temperature at 70±3° C. The slurry was aged for 1 h, cooled to 20±3° C. over 3 h and aged for 1 h. The slurry was filtered and the product washed with iso-propanol (2×700L), methyl tert-butyl ether (560 L) and dried in a vacuum oven at 55° C. to yield the title product (79 kg, 88% th).

¹H NMR (400 MHz, DMSO-D6) δppm 10.5 (2H, s, br); 8.70 (1H, s); 8.22 (1H, d, J=8.1 Hz); 7.86 (1H, d, J=8.1 Hz); 7.36-7.42 (2H, m); 7.15-7.22 (2H, m); 5.43 (2H, s); 2.44 (3H, s).

*KF analysis shows water content of 18.9%; dry weight equivalent 70 kg.

### Steps 4 and 5: 2-(Ethyloxy)-5-(4-{[1-(4-{[(5-methylpyridin-2-yl)methyl]oxy}phenyl)hydrazino]methyl}phenyl)pyridine

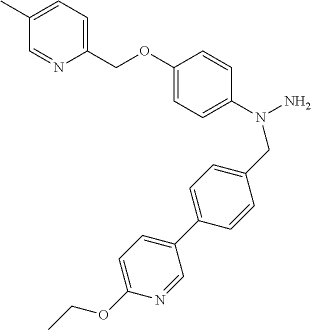

Aqueous sodium nitrite (39.0 kg, 33% w/w) was added at 0-5° C. to a solution of (4-{[(5-methylpyridin-2-yl)methyl]oxy}anilinedihydrochloride (39.0 kg in water 155.8 L) and aqueous hydrogen chloride (conc., 29.6 kg) and washed in with water (7.8 L). This solution was then added at 0-10° C. to a degassed (3×) slurry of sodium hydrosulfite (71 kg) and sodium hydroxide (2.7 kg) in water (155.8 L) followed by a line wash of water (12 L). The resulting mixture was stirred for about 30 min and then warmed to 18±3° C. 2-Propanol (306.5 kg) was added and the pH adjusted to 7.0 using sodium hydroxide (20% w/w, 150.6 kg) maintaining the temperature below 25° C. The layers were allowed to separate and the lower aqueous phase removed. Sodium hydroxide (10% w/w, 78.1 kg) was added followed by 5-[4-(bromomethyl)phenyl]-2-(ethyloxy)pyridine (39.8 kg) and a 2-propanol line wash (3.9 L), and the reaction stirred at 18±3° C. for about 3.5 h. Water (97.5 L) and methanol (195 L) were then added, the mixture stirred for about 12 h and the product isolated by filtration. The crude product was slurry washed with 2-propanol: water (1-1,390 L) followed by two washes with water (2× about 390 kg), then methanol (309 kg) and finally dried at 40-50° C. under vacuum to give the title product (46.45 kg, 77.6% th, ˜93-94% pure by HPLC).

¹H NMR (500 MHz, DMSO-D6) δppm 8.44 (1H, d, J=2.4 Hz); 8.39 (1H, s); 7.97 (1H, dd, J=8.5, 2.7 Hz); 7.62 (1H, dd, J=7.9, 1.5 Hz); 7.59 (2H, d, J=8.2 Hz); 7.37 (3H, t, J=8.5 Hz); 6.97 (2H, d, J=9.2 Hz); 6.82-6.88 (3H, m); 5.02 (2H, s); 4.52 (2H, s); 4.34 (2H, q, J=7.0 Hz); 4.18 (2H, s); 2.29 (3H, s); 1.33 (3H, t, J=7.0 Hz).

### Purification of 2-(ethyloxy)-5-(4-{[1-(4-{[5-methylpyridin-2-yl)methyl]oxy}phenyl)hydrazino]methyl}phenyl)pyridine

2-(Ethyloxy)-5-(4-{[1-(4-{[(5-methyl-2-pyridinyl)methyl]oxy}phenyl)hydrazino]methyl}phenyl)pyridine (50.2 kg) was dissolved in degassed (3×) NMP (210 kg) at 45±3° C., methanol (502 L) was then added maintaining the batch temperature at 43±3° C. and then aged for 30min. The slurry was then cooled to 5±3° C. over about 3 h, aged for 1 h, filtered, washed with methanol (2×198 kg) and then dried at 40-50° C. under vacuum to give the title product (44.9 kg, 89% th).

¹H NMR (500 MHz, DMSO-D6) δppm 8.45 (1H, d, J=2.1 Hz); 8.39 (1H, s) 7.97 (1H, dd, J=8.7, 2.6 Hz); 7.62 (1H, dd, J=8.1, 1.4 Hz); 7.59 (2H, d, J=8.2 Hz); 7.38 (3H, t, J=8.2 Hz); 6.98 (2H, d, J=9.2 Hz); 6.82-6.88 (3H, m); 5.03 (2H, s); 4.53 (2H, s); 4.34 (2H, q, J=7.0 Hz); 4.19 (2H, s); 2.29 (3H, s); 1.34 (3H, t, J=7.0 Hz).

### Steps 4A and 5A: Alternative Synthesis of 2-(ethyloxy)-5-(4-{[1-(4-{[(5-methylpyridin-2-yl)methyl]oxy}phenyl)hydrazino]methyl}phenyl)pyridine

Sodium nitrite (2.47 g) was dissolved in water (10 mL) and added at 0-5° C. to a solution of 4-{[(5-methylpyridin-2-yl)methyl]oxy}aniline dihydrochloride (10 g in water 40 mL) and aqueous hydrogen chloride (conc., 6.4 mL). This solution was then added at <10° C. to a degassed slurry of sodium hydrosulfite (18.2 g) and sodium hydroxide (6.2 mL, 10 wt %) in water (34 mL). The resulting mixture was stirred for 10 min and then warmed to 18±3° C. 2-Propanol (100 mL) was added and the pH adjusted to 7.0 using sodium hydroxide (20 wt %) maintaining the temperature below 25° C. The layers were allowed to separate and the lower aqueous phase removed. Sodium hydroxide (10 wt %, 29 mL) was added followed by 5-[4-(bromomethyl)phenyl]-2-(ethyloxy)pyridine (12.6 g) and the reaction stirrer at 18±3° C. for >2 h. Water (20 mL) and methanol (50 mL) were then added and the product was isolated by filtration. The crude product was then washed with 2-propanol: water (1-1, 100 mL) followed by water (100 mL), and then methanol (100 mL) and finally dried at ca. 45° C. under vacuum to give the title product (11.5 g, 75% th).

¹H NMR (400 MHz, CHLOROFORM-D) δppm 8.42 (1H, s); 8.36 (1H, d, J=2.45 Hz); 7.78 (1H, dd, J=8.56, 2.45 Hz); 7.47-7.54 (3H, m); 7.40 (3H, dd); 7.04-7.10 (2H, m); 6.91-7.00 (2H, m); 6.79 (1H, d, J=8.56 Hz); 5.14 (2H, s); 4.49 (2H, s); 4.40 (2H, q, J=7.09 Hz); 3.51 (2H, s); 2.34 (3H, s); 1.43 (3H, t, J=7.09 Hz).

### Step 4B: Alternative Synthesis of 2-{[(4-hydrazinophenyl)oxy]methyl}-5-methylpyridine dihydrochloride

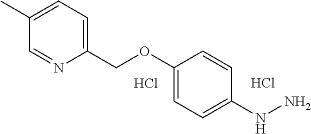

Aqueous sodium nitrite (187.8 g in 0.76 L) was added at 0-5° C. to a solution of (4-{[(5-methylpyridin-2-yl)methyl]oxy}aniline dihydrochloride (760.2 g) and aqueous hydrogen chloride (conc., 487 mL) in water (3.04 L). This solution was then added at <10° C. to a degassed slurry of sodium hydrosulfite (1380 g) and sodium hydroxide (53.2 g) in water (3.04 L).

The resulting mixture was stirred for 30min and then warmed to 18±3° C. The product was extracted in to ethyl acetate (9.5 L) at pH 8-9 using 32% sodium hydroxide. The organic layer was washed with water (2.28 L) and then hydrogen chloride in IPA (5-6 m, 1.29 L) was added over 1 h. The batch was cooled to 5±3° C. over 2h, aged, filtered and the cake washed with IPA (7.6 L), then TBME (5.32 L) and finally dried at 25° C. under vacuum to give the title product (723 g, 90.4% th).

¹H NMR (500MHz, DMSO-D6) δppm 10.22 (3H, s); 8.71 (1H, s); 8.24 (1H, d, J=7.93 Hz); 7.87 (1 H, d, J=8.24 Hz); 7.00-7.06 (4H, m); 5.37 (2H, s); 2.45 (3H, s).

### Step 5B: Alternative Synthesis of 2-(ethyloxy)-5-(4-{[1-(4-{[(5-methylpyridin-2-yl)methyl]oxy}phenyl)hydrazino]methyl}phenyl)pyridine

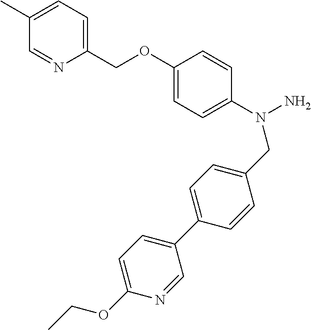

To a slurry of 2-{[4-hydrazinophenyl)oxy]methyl}-5-methylpyridine (400 g) in 2-propanol (3.8 L) was added sodium hydroxide (2M, 2 L) followed by 5-[4-(bromomethyl)phenyl]-2-(ethyloxy)pyridine (380 g) at 18±3° C. After 2 h methanol (2 L) and water (2 L) were added and the slurry cooled to 5±3° C. The slurry was filtered and washed with methanol (2 L), water (2 L), then finally methanol (3 L) before being dried at 45-55° C. under vacuum to give the title product (505 g, 87% th).

### Purification of 2-(ethyloxy)-5-(4-{[1-(4-{[(5-methylpyridin-2-yl)methyl]oxy}phenyl)hydrazino]methyl}phenyl)pyridine

The crude product, 2-(ethyloxy)-5-(4-{[1-(4-{[(5-methylpyridin-2-yl)methyl]oxy}phenyl)hydrazino]methyl}phenyl)pyridine (495 g), was dissolved in degassed NMP (1.98 L) at 45±3° C., methanol (4.95 L) was then added maintaining the temperature at 40-48° C. After 30 min the slurry was cooled to 5±3° C. over 2 h, aged for 1 h and then filtered and washed with methanol (2×2.5L) then dried at 40-50° C. under vacuum to give the title product (411 g, 82.9%).

¹H NMR (400 MHz, DMSO-D6) δppm 8.45 (1H, d, J=2.69 Hz); 8.39 (1H, d, J=2.20 Hz); 7.98 (1H, dd, J=8.68, 2.57 Hz); 7.62 (1H, dd, J=8.31, 1.96 Hz); 7.59 (2H, d, J=8.31 Hz); 7.38 (3H, t, J=7.46 Hz); 6.95-7.00 (2H, m); 6.84-6.88 (3H, m); 5.03 (2H, s); 4.53 (2H, s); 4.34 (2H, q, J=6.93 Hz); 4.20 (2H, s); 2.30 (3H, s); 1.34 (3H, t, J=7.09 Hz).

### Steps 4B and 5B: Alternative Synthesis of 2-(ethyloxy)-5-(4-{[1-(4-{[(5-methylpyridin-2-yl)methyl]oxy}phenyl)hydrazino]methyl}phenyl)pyridine

Sodium nitrite (0.19 kg) was dissolved in water (0.8 L) and added at 0-5° C. to a solution of 4-{[(5-methylpyridin-2-yl)methyl]oxy}aniline dihydrochloride (0.80 kg) in water (3.2 L) and aqueous hydrogen chloride (conc., 0.51 L), washing in with further water (0.4 L). This solution was then added at 0±3° C. to a degassed (3×) slurry of sodium hydrosulfite (1.46 kg) and potassium hydroxide (0.080 kg) in water (3.2 L), washing in with further water (1.2 L). 2-Propanol (4 L), potassium hydroxide (1.10 kg) and 5-[4-(chloromethyl)phenyl]-2-(ethyloxy)pyridine (0.67 kg) were added and the reaction heated to 45±5° C. for ca. 4 h. Volatiles (ca. 4 L) were removed by distillation under vacuum and 2-methyltetrahydrofuran (9.6 L) was added. The reaction was heated to 63±3° C. and the lower aqueous phase removed. The organic phase was washed with water (3.2 L) at 63±3° C., then 2-propanol (9.6 L) was added at 55±5° C. The resulting slurry was cooled to 20±3° C. and the solids collected by filtration. The filter cake was washed twice with 2-propanol (4 L) and dried at ca. 45° C. under vacuum to give the title product (0.953 kg, 78% th.) with >99% area purity by HPLC.

¹H NMR (400 MHz, CHLOROFORM-D) δppm 8.42 (1H, s); 8.36 (1H, d, J=2.45 Hz); 7.78 (1H, dd, J=8.56, 2.45 Hz); 7.47-7.54 (3H, m); 7.40 (3H, dd); 7.04-7.10 (2H, m); 6.91-7.00 (2H, m); 6.79 (1H, d, J=8.56 Hz); 5.14 (2H, s); 4.49 (2H, s); 4.40 (2H, q, J=7.09 Hz); 3.51 (2H, s); 2.34 (3H, s); 1.43 (3H, t, J=7.09 Hz).

### Step 6: ethyl 3-[3-(tert-butylsulfanyl)-1-[4-(6-ethoxy-pyridin-3-yl)benzyl]-5-(5-methylpyridin-2-yl)methoxy)-1H-indol-2-yl]-2,2-dimethyl-propanoate

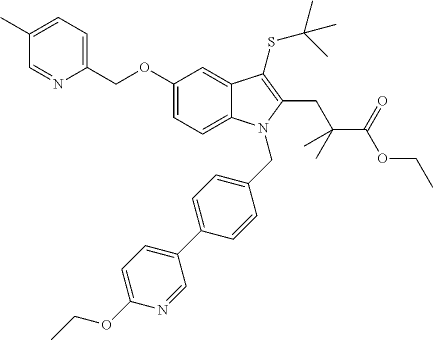

To a degassed slurry of 2-(ethyloxy)-5-(4-{[1-(4-{[(5-methylpyridin-2-yl)methyl]oxy}phenyl)hydrazino]methyl}phenyl)pyridine (43.0 kg) and ethyl 5-[(1,1-dimethylethyl)thio]-2,2-dimethyl-4-oxopentanoate (39.6 kg) in 2-propanol (344 L) was added a degassed slurry of dibenzoyl tartaric acid monohydrate (147.1 kg) in 2-propanol (344 L). The reaction was stirred at 25±3° C. for 6 h and then at 45±3° C. for 10 h. After this period the reaction was concentrated by atmospheric distillation to 731 L, and water (172 L) added. The reaction was clarified through a bed of celite and the celite washed with 2-propanol (86 L), water was added (86 L) before a further concentration to 989 L. The solution was seeded at 65±3° C. and cooled to 20±3° C. before filtration. The filter cake was washed with 2-propanol:water (2:1, 426 L) followed by ethanol (427 L) and then dried at 45-55° C. under vacuum to give the title product (48.7 kg, 75% th).

¹H NMR (500 MHz, CHLOROFORM-D) δppm 8.42 (1H, s); 8.30 (1H, d, J=2.1 Hz); 7.69 (1H, dd, J=8.5, 2.4 Hz); 7.43-7.48 (2H, m); 7.38 (2H, d, J=8.2 Hz); 7.35 (1 H, d, J=2.1 Hz); 7.09 (1 H, d, J=8.9 Hz); 6.85-6.91 (3H, m); 6.74 (1 H, d, J=8.5 Hz); 5.42 (2H, s); 5.24 (2H, s); 4.37 (2H, q, J=7.1 Hz); 4.06 (2H, q, J=7.1 Hz); 3.32 (2H, s); 2.30 (3H, s); 1.39 (3H, t, J=7.0 Hz); 1.24 (15H, s); 1.17 (3H, t, J=7.2 Hz).

### Step 6A: Alternative Synthesis of ethyl 3-[3-(tert-butylsulfanyl)-1-[4-(6-ethoxy-pyridin-3-yl)benzyl]-5-(5-methylpyridin-2-yl)methoxy)-1H-indol-2-yl]-2,2-dimethyl-propanoate

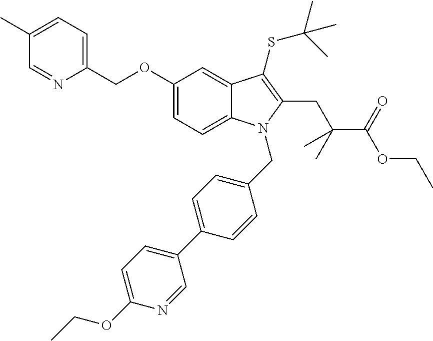

2-(Ethyloxy)-5-(4-{[1-(4-{[(5-methylpyridin-2-yl)methyl]oxy}phenyl)hydrazino]methyl}phenyl)pyridine (70 g) and ethyl 5-[(1,1-dimethylethyl)thio]-2,2-dimethyl-4-oxopentanoate (62 g) were stirred in a mixture of ethanol (70 mL) and isobutyric acid (490 mL). The slurry was degassed and heated at 23-25° C. for 12 h and then heated to 40±3° C. over 12 h. After a further 9 h at this temperature the reaction was heated to 70° C. and ethanol (280 mL) added followed by water (490 mL). The temperature was then adjusted 75° C. and seeded. The resulting suspension was cooled to 5° C. and the product isolated by filtration. The solid was washed with ethanol (2×350 mL) at 5° C. and dried at 40° C. under vacuum to give the title product (76 g, 72% th).

¹H NMR (400 MHz, DMSO-D6) δppm 8.39-8.42 (2H, m); 7.94 (1 H, dd, J=8.6, 2.4 Hz); 7.60 (1H, dd, J=7.9, 2.1 Hz); 7.55 (2H, d, J=8.3 Hz); 7.38 (1H, d, J=7.8 Hz); 7.30 (1H, d, J=9.0 Hz); 7.10 (1H, d, J=2.4 Hz); 6.90 (2H, d, J=8.1 Hz); 6.83 (2H, d, J=8.6 Hz); 5.50 (2H, s); 5.15 (2H, s); 4.32 (2H, q, J=7.0 Hz); 4.04 (2H, q, J=7.1 Hz); 3.25 (2H, s); 2.28 (3H, s); 1.32 (3H, t, J=7.1 Hz); 1.11-1.17 (18H, m).

### Step 7: 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid

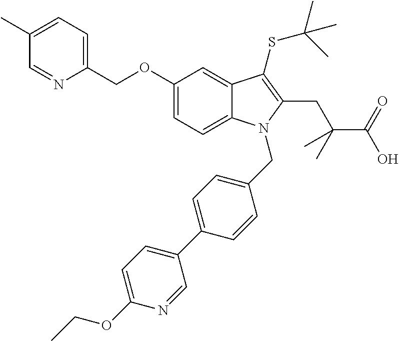

To a suspension of ethyl 3-[3-(tert-butylsulfanyl)-1-[4-(6-ethoxy-pyridin-3-yl)benzyl]-5-(5-methylpyridin-2-yl)methoxy)-1H-indol-2-yl]-2,2-dimethyl-propanoate (47.56 kg) in tetrahydrofuran (71 L) was added ethanol (41.8 L) and aqueous sodium hydroxide (46-48 wt %, 10.46 kg). The reaction was then heated at reflux for 1-2 h before cooling to 20±3° C. and clarified. The filter was washed with tetrahydrofuran (24 L) and the solution was then acidified with hydrochloric acid (2M) to pH 4. Water (143 L) was then added and the slurry cooled to 2±3° C. before isolation of the product by filtration. The filter cake was washed with 2:1 water:tetrahydrofuran (142.5 L) at 2±3° C. followed by ethyl acetate (143 L) and dried at 45-55° C. under vacuum to give the title product (44.5 kg, 97.7%).

¹H NMR (400 MHz, DMSO-D6) δppm 8.39-8.44 (2H, m); 7.95 (1H, dd, J=8.7, 2.6 Hz); 7.61 (1H, dd, J=7.9, 1.3 Hz); 7.55 (2H, d, J=8.3 Hz); 7.40 (1H, d, J=7.8 Hz); 7.34 (1H, d, J=8.8 Hz); 7.13 (1H, d, J=2.2 Hz); 6.91 (2H, d, J=8.1 Hz); 6.81-6.87 (2H, m); 5.53 (2H, s); 5.16 (2H, s); 4.33 (2H, q, J=6.9 Hz); 3.24 (2H, s); 2.30 (3H, s); 1.33 (3H, t, J=7.0 Hz); 1.10-1.18 (15H, m).

### Purification of 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid

3-[3-(tert-Butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid (1.35 kg) was dissolved in 2-butanone (20.0 L) and water (0.9 L volumes) at 75±3° C. The solution was cooled to 65° C. and filtered. The transfer lines were washed with 2-butanone (1.35 L) and the combined filtrate and wash concentrated by distillation at atmospheric pressure to leave a residual volume of 10 Lvolumes. The suspension was cooled to 0±3° C. and stirred for 1 h at this temperature. The product was collected by filtration, washed with 2-butanone (5.4 L) then ethyl acetate (2.7 L) and dried under vacuum at 50±5° C. to give the title product (1.26 kg, 94% th).

¹H NMR (400 MHz, DMSO-D6) δppm 12.46 (1 H, br s); 8.39-8.44 (2H, m); 7.94 (1 H, dd, J=8.7, 2.6 Hz); 7.60 (1H, dd, J=7.9, 1.3 Hz); 7.54 (2H, d, J=8.3 Hz); 7.39 (1H, d, J=7.8 Hz); 7.33 (1H, d, J=8.8 Hz); 7.12 (1H, d, J=2.2 Hz); 6.91 (2H, d, J=8.2 Hz); 6.81-6.87 (2H, m); 5.52 (2H, s); 5.16 (2H, s); 4.32 (2H, q, J=6.9 Hz); 3.24 (2H, s); 2.39 (3H, s); 1.33 (3H, t, J=7.0 Hz); 1.14 (9H, s); 1.12 (6H, s).

### Step 6B & 7A: 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid

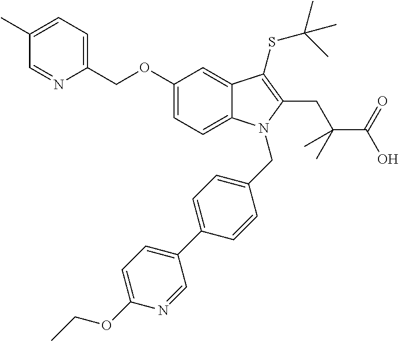

To a degassed slurry of 2-(ethyloxy)-5-(4-{[1-(4-{[(5-methylpyridin-2-yl)methyl]oxy}phenyl)hydrazino]methyl}phenyl)pyridine (1.0 kg) and ethyl 5-[(1,1-dimethylethyl)thio]-2,2-dimethyl-4-oxopentanoate (720 mL) in 2-MeTHF (4.0 L) was added dibenzoyl tartaric acid monohydrate (854 g), citric acid (436 g) and 2-MeTHF (1 L). The reaction was stirred at 30±2° C. for 6 h and then heated to 55±2° C. and held at this temperature until the reaction was complete. Water (3.25 L) and 10 wt % sodium hydroxide (3.25 L) was added to achieve pH 7 then the lower aqueous layer was discarded. The reaction was then concentrated by atmospheric distillation to 3.4 L, and 2-propanol (8.7 L) wad added. Sodium hydroxide (236 g) was added and the mixture heated at reflux for ca.4 h before cooling to 67±3° C. The solution was then acidified with hydrochloric acid (2.6 L, 2M) to pH 6. After ageing, water (0.8 L) was added and the slurry cooled to 45±3° C. before isolation of the product by filtration. The filter cake was washed with 1.0:3.5:1.5 2-MeTHF:2-propanol:water (6.0 L), then by 2-propanol (6 L) and dried at 45° C. under vacuum to give the title product (1.06 kg, 73%).

¹H NMR (400 MHz, DMSO-D6) δppm 8.1-8.43 (2H, m); 7.95 (1 H, dd, J=8.7, 2.6 Hz); 7.61 (1H, dd, J=7.8, 1.3 Hz); 7.55 (2H, d, J=8.3 Hz); 7.40 (1H, d, J=7.8 Hz); 7.34 (1H, d, J=8.8 Hz); 7.13 (1H, d, J=2.4 Hz); 6.91 (2H, d, J=8.1 Hz); 6.83-6.86 (2H, m); 5.53 (2H, s); 5.16 (2H, s); 4.33 (2H, q, J=6.9 Hz); 3.24 (2H, s); 2.31 (3H, s); 1.33 (3H, t, J=7.0 Hz); 1.11-1.16 (15H, m).

### Step 6C & 7B: 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid

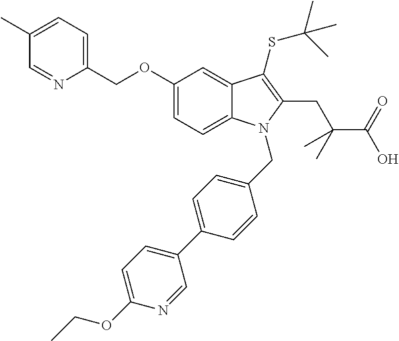

2-(Ethyloxy)-5-(4-{[1-(4-{[(5-methylpyridin-2-yl)methyl]oxy}phenyl)hydrazino]methyl}phenyl)pyridine (46.0 kg) and ethyl 5-[(1,1-dimethylethyl)thio]-2,2-dimethyl-4-oxopentanoate (32.3 kg) were added to degassed (4×) 2-MeTHF (151 kg) and were washed in with 2-MeTHF (20 Kg) The degassing was then repeated (4×). Dibenzoyl tartaric acid monohydrate (39.3 kg) and citric acid (20.1 kg) were then added followed by a 2-MeTHF (22 kg) line rinse and the mixture degassed again (4×). The reaction was stirred at 30±2° C. for about 6 h and then heated to 55±2° C. and held at this temperature until the reaction was complete (about 15 h). Water (152 kg) and 10 wt % sodium hydroxide (167 kg) was added and the mixture stirred for about 1 h and then allowed to settle, the lower aqueous layer was discarded at 50±2° C. The reaction was then concentrated by atmospheric distillation to ˜155L. 2-Propanol (290 kg) and sodium hydroxide (10.6 kg) were added and the mixture heated at reflux until the reaction was complete (about 15 h). After cooling to 65-70° C., the solution was diluted with 2-propanol (32 kg) then neutralised with hydrochloric acid (123 kg, 2M). Water (55 L) was added and the slurry cooled to 42-45° C. and aged for about 4 h before the product was isolated by filtration. The filter cake was washed with 2-MeTHF:2-propanol:water (19.5 kg:64 kg:34 kg), then by 2-propanol (217 kg) and dried under vacuum to give the title product (50.4 kg, 76%).

¹H NMR (400 MHz, DMSO-D6) δppm 8.38-8.43 (2H, m); 7.93 (1 H, dd, J=8.6, 2.7 Hz); 7.59 (1H, dd, J=8.0, 1.6 Hz); 7.54 (2H, d, J=8.12 Hz); 7.38 (1H, d, J=7.9 Hz); 7.32 (1H, d, J=8.9 Hz); 7.11 (1 H, d, J=2.2 Hz); 6.90 (2H, d, J=8.4 Hz); 6.80-6.86 (2H, m); 5.51 (2H, s); 5.15 (2H, s); 4.32 (2H, q, J=7.1 Hz); 3.24 (2H, s); 2.28 (3H, s); 1.31 (3H, t, J=7.0 Hz); 1.07-1.16 (15H, m)

### Step 8: Sodium 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionate

### Polymorph Form C

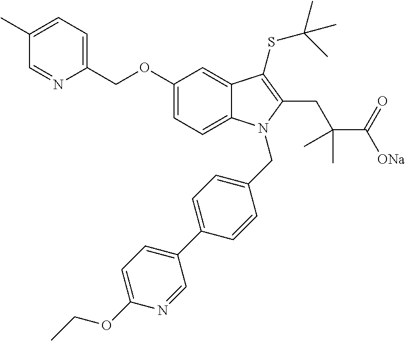

3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid (28.3 kg) was dissolved in ethanol (32.6 Kg) by the addition of sodium hydroxide (3.7 kg, 46-48%) in ethanol (8.5 L) and heating at 72° C. for about 25 min. The resulting solution was cooled to 55±3° C., diluted with diisopropylether (78 L), seeded with sodium 3[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionate Polymorph Form C (28 g) and stirred for about 1 hr. Further diisopropylether (280 L) was added and the contents were stirred at 55±3° C. for about 1 h. The contents were then cooled to 20±3° C. and stirred overnight (about 11 hours). The slurry was allowed to settle for ca 10 min before being filtered under nitrogen. The filter cake was washed with diisoproyl ether:ethanol (9:1, 84.5 L) followed by diisopropyl ether (85 L) and dried at 45-55° C. under vacuum to give the title product (25.75 kg, 88.0% th).

¹H NMR (500 MHz, DMSO-D6) δppm 8.38-8.41 (2H, m); 7.93 (1 H, dd, J=8.54, 2.75 Hz); 7.59 (1H, dd, J=7.93, 1.53 Hz); 7.51 (2H, d, J=8.24 Hz); 7.38 (1H, d, J=7.93 Hz); 7.22 (1H, d, J=8.85 Hz); 7.08 (1H, d, J=2.44 Hz); 6.92 (2H, d, J=8.24 Hz); 6.82 (1H, d, J=8.54 Hz); 6.76 (1H, dd, J=8.85, 2.44 Hz); 5.67 (2H, s); 5.13 (2H, s); 4.31 (2H, q, J=7.02 Hz); 3.20 (2H, s); 2.28 (3H, s); 1.31 (3H, t, J=7.02 Hz); 1.13 (9H, s); 0.97 (6H, s).

### Step 8A: Sodium 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionate

### Polymorph Form C

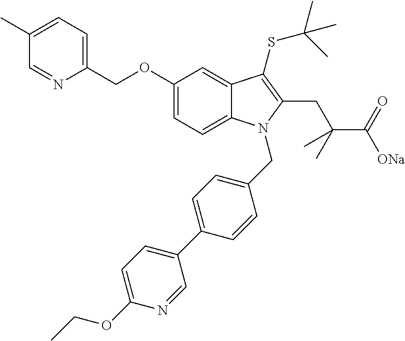

3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionic acid (2.43 g, 3.8 mmol, 0.97 wt), sodium hydroxide pellets (0.17 g, 4.4 mmol, 0.0697 wt) and TBME (8.75 ml, 3.5 vol) were charged into a vessel. The resulting slurry was heated to 50° C. over 10 min with stirring. After a further 35 min, methanol (3.75 ml, 1.5vol) was added and the slurry aged at 50° C. for 45 min. A solution was formed and 7:3 TBME:methanol (2.5 ml, 1 vol) was added to the vessel to simulate a line wash. TBME (7.5 ml, 3 vol) was charged to the vessel over 30 min. The solution was then seeded with a slurry of sodium 3-[3-(tert-butylsulfanyl)-1-[4-(6-ethoxy-pyridin-3-yl)benzyl]-5-(5-methyl-pyridin-2-yl-methoxy)-1H-indol-2-yl]-2,2-dimethyl-propionate Polymorph Form C (0.025 g, 0.038 mmol, 0.01 wt) in TBME (0.5 ml, 0.2 vol). The resulting slurry was aged at 50° C. for 1 h 15 min and then TBME (22.5 ml, 9 vol) was added over 1 h. The slurry was aged for a further hour at 50° C., filtered and washed with TBME (2×10 ml) and then dried at 50° C. in vacuo.

¹H NMR (400 MHz, MeOH) δppm 8.36 (1H, s); 8.26 (1H, d, J=2.45 Hz); 7.85 (1H, dd, J=8.68, 2.57 Hz); 7.65 (1H, d, J=8.07 Hz); 7.47 (1H, d, J=8.07 Hz); 7.41 (2H, d, J=8.07 Hz); 7.12-7.17 (2H, m); 6.93 (2H, d, J=8.31 Hz); 6.77-6.83 (2H, m, J=8.74, 2.48, 2.48 Hz); 5.61 (2H, s); 5.17 (2H, s); 4.31 (2H, q, J=7.09 Hz); 2.34 (3H, s); 1.37 (3H, t, J=7.09 Hz); 1.17 (9H, s); 1.12 (6H, s).

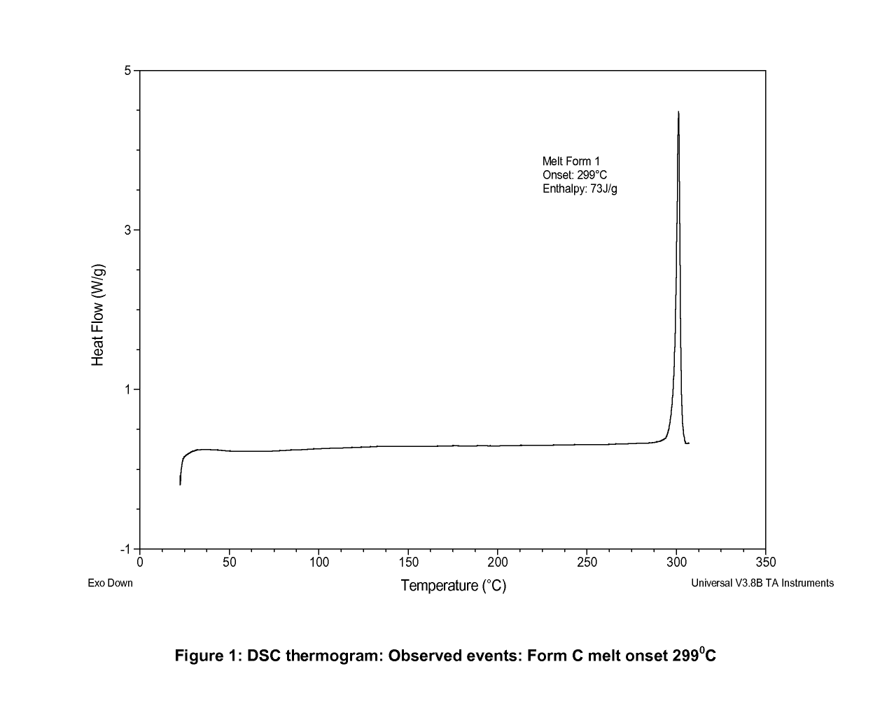

DSC thermogram of the title product is shown in FIG. 1.

The DSC thermogram was obtained using a TA Q2000 calorimeter. The sample was weighed into an aluminium pan and a pan lid pushed on top without sealing the pan. The experiment was conducted using a heating rate of 10° C. min⁻¹.

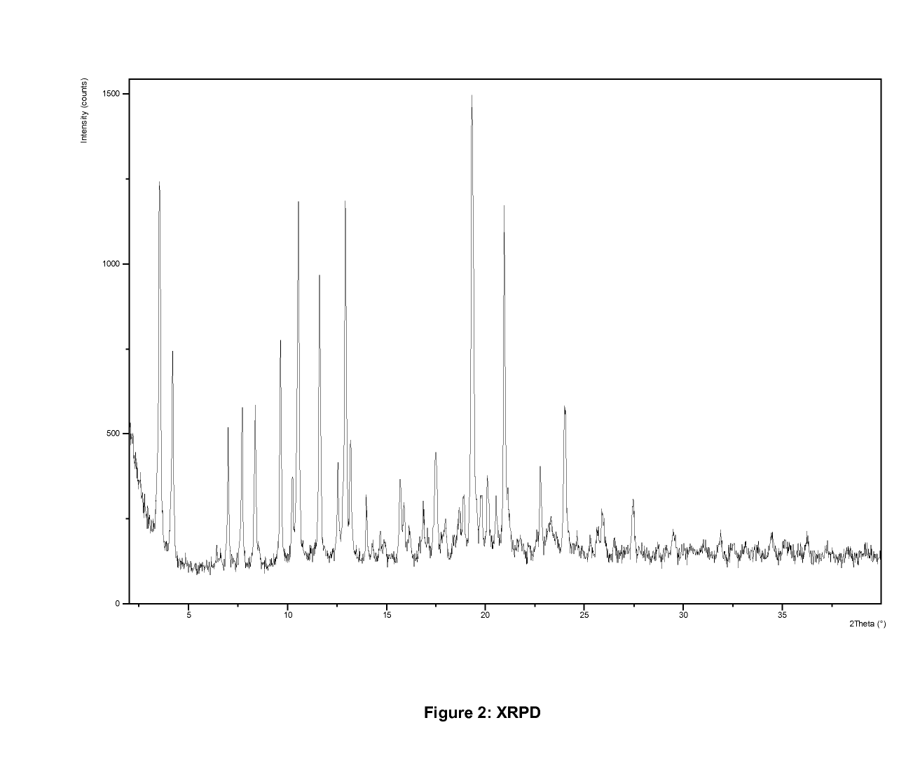

XRPD profile of the title product is shown in FIG. 2.

The data was acquired on a PANalytical X′Pert Pro powder diffractometer using an XCelerator detector. The acquisition conditions were: radiation: Cu Kα, generator tension: 40 kV, generator current: 45 mA, start angle: 2.0° 2θ, end angle: 40.0° 2θ, step size: 0.0167° 2θ, time per step: 31.75 seconds. The sample was prepared by mounting a few milligrams of sample on a Si wafer (zero background) plate, resulting in a thin layer of powder. Characteristic XRPD angles and d-spacings are recorded in Table 1. The margin of error is approximately±0.1° 2θfor each of the peak assignments. Peak intensity may vary from sample to sample due to preferred orientation.

Peak positions were measured using Highscore software.

TABLE 1
Characteristic XRPD peak positions and d-spacings

| 2θ/° | d-spacing/Å |
|---|---|
| 3.5 | 25.0 |
| 4.2 | 21.0 |
| 7.0 | 12.6 |
| 7.7 | 11.5 |
| 8.4 | 10.6 |
| 9.6 | 9.2 |
| 10.5 | 8.4 |
| 11.6 | 7.6 |
| 12.9 | 6.8 |
| 17.5 | 5.1 |
| 19.3 | 4.6 |
| 20.9 | 4.2 |
| 24.0 | 3.7 |

What is claimed is:

1. A process for preparing a compound of formula (II) 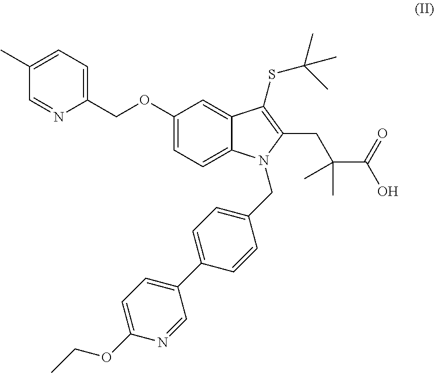
or a salt thereof;
comprising reacting a compound of formula (VII) 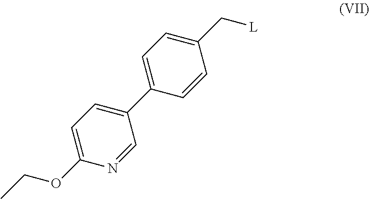
or a salt thereof;
wherein L is a leaving group;
with a compound of formula (VI) 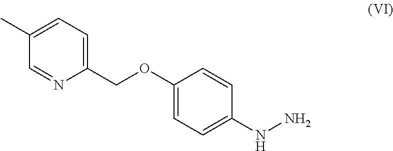
or a salt thereof;
in the presence of a base and solvent, and then converting to a compound of formula (II) or a salt thereof.

2. A telescoped process for preparing a compound of formula (II) 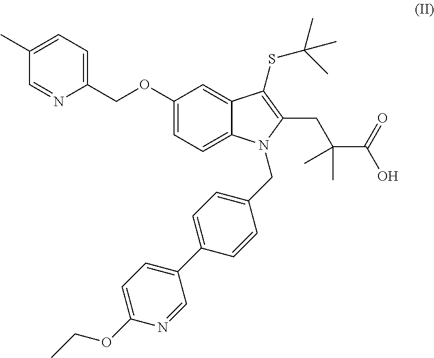
or a salt thereof;
comprising a process for preparing a compound of formula (VI) comprising reacting a compound of formula (VIII) 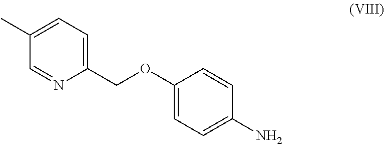
or a salt thereof;
with aqueous sodium nitrite in the presence of hydrochloric acid to form the diazonium salt followed by reduction of the diazonium salt;
followed by a process for preparing a compound of formula (IVa) comprising reacting a compound of formula (VII) 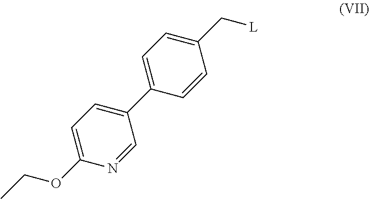
or a salt thereof;
wherein L is a leaving group;
with a compound of formula (VI) 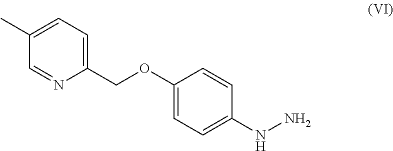
or a salt thereof;
in the presence of a base and solvent
wherein the compound of formula (VI) is not isolated, and then converting to a compound of formula (II).

3. A process according to claim 1 for preparing a compound of formula (II).

4. A process according to claim 1 for preparing a salt of a compound of formula (II).

5. (canceled)

6. (canceled)

7. A process according to claim 1 wherein L is selected from chlorine and bromine.

8. A process according to claim 7 wherein L is chlorine.

9. A process according to claim 8 wherein the chlorinating agent is added at ≦20° C. and the mixture then heated at from about 20° C. to about 35° C.

10. A process according to claim 1 wherein the base is selected MOH, M₂CO₃ and MHCO₃ wherein M is selected from Li (lithium), Na (sodium), K (potassium) and Cs (caesium); 1,8-diazabicyclo[5.4.0]undec-7-ene; and R′R″R″′N wherein R′, R″ and R″′ are each independently C₁-C₆alkyl.

11. A process according to claim 10 wherein the base is MOH.

12. A process according to claim 11 wherein the base is KOH.

13. A process according to claim 1 wherein the solvent is selected from C₁-C₆alcohol, tetrahydrofuran, 2-methyltetrahydrofuran, toluene, dichloromethane and mixtures thereof.

14. A process according to claim 13 wherein the solvent is selected from ethanol, 1-propanol, 2-propanol, 2-butanol, sec-butanol and mixtures thereof.

15. A process according to claim 1 wherein the compound of formula (VII) is in the form of the free base.

16. A process according to claim 1 wherein the compound of formula (VII) is in the form of a salt.

17. A process according to claim 16 wherein the salt of the compound of formula (VII) is selected from hydrogen bromide, hydrogen chloride, hydrogen iodide, p-toluenesulfonate, methanesulfonate, trifluoromethanesulfonate and phosphate.

18. A process according to claim 1 wherein the compound of formula (VI) is in the form of the free base.

19. A process according to claim 1 wherein the compound of formula (VI) is in the form of a salt.

20. A process according to claim 19 wherein the salt of the compound of formula (VI) is the dihydrogen chloride salt.

21. A process for preparing the anhydrous Form C polymorph of a compound of formula (I) 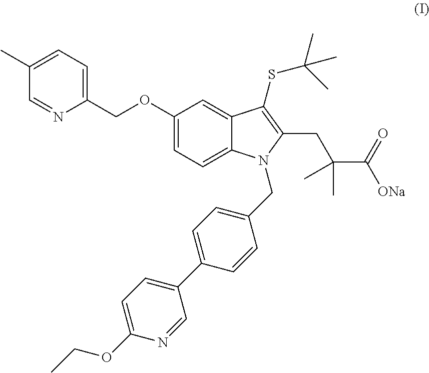
comprising dissolving a compound of formula (II) 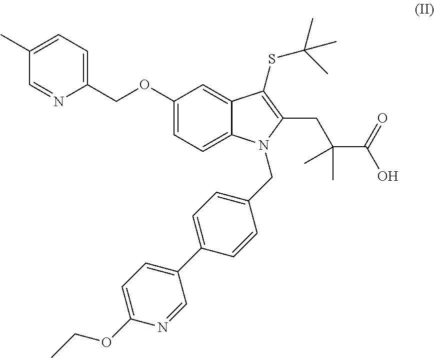
in methanol and methyl-t-butylether in the presence of solid sodium hydroxide, followed by addition of methyl-t-butylether, wherein the solvent system in the reactant mixture contains 30% or less methanol by volume.

22-59. (canceled)
